(19) United States
(12) Patent Application Publication
(10) Pub. No.: US 2021/0238685 A1
(43) Pub. Date: Aug. 5, 2021

(54) BIOMARKER FOR CONFIRMING DEGENERATIVE DISEASE TREATMENT OF DROSOPHILA USING LOW-DOSE RADIATION

(71) Applicant: KOREA HYDRO & NUCLEAR POWER CO., LTD., Gyeongsangbuk-do (KR)

(72) Inventors: Seonyoung NAM, Seoul (KR); Soojin HWANG, Seoul (KR); Haemin JEONG, Gyeonggi-do (KR); Eunhee HONG, Seoul (KR)

(21) Appl. No.: 17/052,827

(22) PCT Filed: May 15, 2019
(86) PCT No.: PCT/KR2019/005816

§ 371 Date: Nov. 4, 2020

(30) Foreign Application Priority Data
May 16, 2018 (KR) 10-2018-0056068

(51) Int. Cl. C12Q 1/6883

(52) U.S. Cl. CPC C12Q 1/6883; C12Q 2600/158

(57) ABSTRACT

A biomarker for confirming the treatment of a Drosophila degenerative disease is disclosed, including a gene involved in immune response activation, a gene involved in neuronal regeneration, and a gene involved in motility function. All of these are sensitive to low-dose radiation, and, more particularly to classification and selection of genes responsive when treating degenerative diseases of Drosophila using low-dose radiation. The disclosed genes exhibiting changes in expression upon low-dose radiation are classified depending on the function thereof, and response marker genes responsive to low-dose radiation are provided. These genes can thus be effectively utilized as basic data for developing a therapeutic marker when applying low-dose radiation for the treatment of degenerative diseases.

### TECHNICAL FIELD

The present disclosure relates to a technique for providing in-vivo models and markers that are useful in verifying the efficacy and safety of Alzheimer's disease treatment using low-dose radiation and are additionally useful as basic data for the development of therapeutic markers when applying low-dose radiation for the treatment of degenerative diseases.

### BACKGROUND ART

Recently, low-dose radiation has emerged as a means of treating degenerative diseases, and as for Drosophila, a Drosophila model (elav>Aβ42) for Alzheimer's disease, a kind of degenerative disease, which is usefully employed in various studies, is provided and is utilized to study the onset and treatment mechanisms of Alzheimer's disease and to test therapeutic agents.

Currently, a Drosophila degenerative disease model is constructed as an in-vivo model capable of testing the therapeutic effect of low-dose radiation before clinical trials, but there is no research on response gene markers specific to low-dose radiation related to the process of alleviating degenerative disease symptoms.

Accordingly, there is a need to develop response marker genes for low-dose radiation from the Alzheimer's disease Drosophila model, and to construct related basic data in in-vivo models for clinical radiation therapy tests and the like in the future.

### CITATION LIST

### Patent Literature

(Patent Document 1) Korean Patent Application Publication No. 10-2017-0083704

### DISCLOSURE

### Technical Problem

An objective of the present invention is to provide genes, in which the head tissue of adult Drosophila raised at different cumulative dose rates is separated and the entire gene expression change pattern thereof is analyzed through an RNA sequencing method, thereby enabling the use thereof for the development of an in-vivo model and a marker to verify the efficacy and safety of Alzheimer's disease treatment using low-dose radiation.

Another objective of the present invention is to provide response marker genes responsive to low-dose radiation by classifying genes, the expression of which is changed due to low-dose radiation, depending on the function thereof, thereby making it possible to use the same as basic data for the development of therapeutic markers when applying low-dose radiation for the treatment of degenerative diseases.

### Technical Solution

In order to accomplish the above objectives, the present disclosure provides a biomarker responsive when treating a Drosophila degenerative disease using low-dose radiation.

The low-dose radiation is preferably applied so that a cumulative dose is 0.01 to 0.2 Gy.

The Drosophila may be an Alzheimer's disease Drosophila model. Also, the Alzheimer's disease Drosophila model may be GMR>Aβ42 or elav>Aβ42.

The degenerative disease may be Alzheimer's disease.

The biomarker may include a gene involved in immune response activation, a gene involved in neuronal regeneration, a gene involved in motility function, and the like. Here, examples of the gene involved in immune response activation may include PGRP-SC1a and PGRP-SC1b, examples of the gene involved in neuronal regeneration may include en, Oseg4, PCNA, and scra, and examples of the gene involved in motility function may include Gas8 and TrpA1. In addition, CG9272, ECSIT and the like may be included in the biomarker.

### Advantageous Effects

According to the present invention, there are provided genes, in which the head tissue of adult Drosophila raised at different cumulative dose rates is separated and the entire gene expression change pattern thereof is analyzed through an RNA sequencing method, thereby enabling the use thereof for the development of an in-vivo model and a marker to verify the efficacy and safety of Alzheimer's disease treatment using low-dose radiation.

In addition, there are provided response marker genes responsive to low-dose radiation by classifying genes, the expression of which is changed due to low-dose radiation, depending on the function thereof, thereby making it possible to use the same as basic data for the development of therapeutic markers when applying low-dose radiation for the treatment of degenerative diseases.

### DESCRIPTION OF DRAWINGS

FIG. 1a shows the results of comparison of the symptom alleviation effects depending on the dose of low-dose radiation in Drosophila Alzheimer's disease models, and FIG. 1b shows the results of comparison of the symptom alleviation effects depending on the dose rate of low-dose radiation in Drosophila Alzheimer's disease models;

FIG. 2 shows gene groups specifically responding to low-dose radiation, classified depending on the function thereof, in Alzheimer's disease Drosophila models;

FIG. 3 shows gene lists specifically responding to low-dose radiation, which exhibit changes in gene expression for relieving Alzheimer's disease; and

FIG. 4 shows changes in the expression of the genes shown in FIG. 3 through real-time PCR.

### BEST MODE

Hereinafter, a detailed description will be given of the present disclosure.

An aspect of the present disclosure pertains to a biomarker for confirming the treatment of a Drosophila degenerative disease, including a gene involved in immune response activation, a gene involved in neuronal regeneration, and a gene involved in motility function, all of which are sensitive to low-dose radiation.

The low-dose radiation is preferably applied so that the cumulative dose is 0.01 to 0.2 Gy, and it is preferable to realize a cumulative dose of 0.01 to 0.2 Gy at different dose rates (¹³Cs, 15.936 mGy/s, 3.147 mGy/s, 5 mGy/h). It is more preferable to apply low-dose radiation so that the cumulative dose is 0.05 Gy. In accordance with UNSCEAR (United Nations Scientific Committee on the Effects of Atomic Radiation), a cumulative dose of 100 mGy or less is defined as a low dose. The standard for a low dose in the present disclosure follows the standard of UNSCEAR.

The Drosophila is an Alzheimer's disease Drosophila model. The Alzheimer's disease Drosophila model is GMR>Aβ42 or elav>Aβ42. The GMR>Aβ42 and elav>Aβ42 are Alzheimer's disease Drosophila models. The Alzheimer's disease Drosophila model was obtained by overexpressing Aβ42 (using UAS-Aβ42), a gene causative of Alzheimer's disease, specifically in the Drosophila nerve (neuron; using elav-GAL4) using the UAS/GAL4 system, and was used in the experiment. Moreover, in order to determine the dose for use in RNA sequencing, the Alzheimer's disease Drosophila model was obtained by overexpressing Aβ42 (using UAS-Aβ42) specifically in the Drosophila eye (using GMR-GAL4), and a phenotype in which the size of the eye is decreased was observed when overexpressing Aβ42 in the Drosophila eye.

The degenerative disease is Alzheimer's disease. Alzheimer's disease is the leading cause of dementia, which occurs mainly in the elderly. The cause of Alzheimer's disease has been found to be highly associated with beta-amyloid protein. When beta-amyloid is made excessively in the body and accumulates in brain cells, the function of the brain neurons decreases, resulting in Alzheimer's disease. Beta-amyloid paralyzes or distorts the function of mitochondria in the neurons, thereby increasing the amount of reactive oxygen species released from mitochondria. The reactive oxygen species thus increased inflict fatal damage to proteins or DNA in cells, leading to damage to brain cells or apoptosis.

In order to confirm variation in symptom alleviation effects depending on the dose and dose rate of radiation through the analysis of genes responsive when treating the degenerative disease, Drosophila embryos for 0-6 hr irradiated with low-dose radiation were raised, the head tissue of adult Drosophila was separated, and the entire gene expression change pattern thereof was investigated through an RNA sequencing method, so genes exhibiting changes in expression due to low-dose radiation were analyzed.

The biomarker includes a gene involved in immune response activation, a gene involved in neuronal regeneration, a gene involved in motility function, and the like, and representative examples of the gene involved in immune response activation may include PGRP-SC1a and PGRP-SC1b, and representative examples of the gene involved in neuronal regeneration may include en, Oseg4, PCNA, and scra. Also, representative examples of the gene involved in motility function may include Gas8 and TrpA1. In addition, CG9272, ECSIT and the like may also be included in the biomarker.

A better understanding of the present disclosure may be obtained through the following examples. These examples are merely set forth to illustrate the present disclosure, but are not to be construed as limiting the scope of the present disclosure, which will be apparent to those skilled in the art.

### Example 1. Symptom Alleviation Effect Depending on Dose and Dose Rate of Low-Dose Radiation in Drosophila Alzheimer's Disease Model

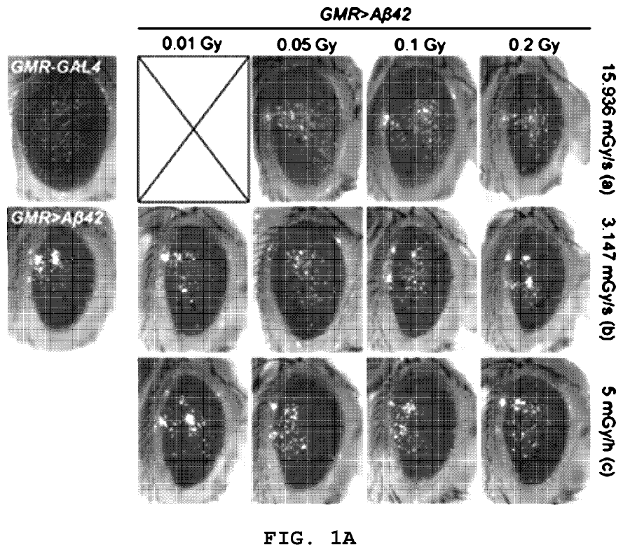

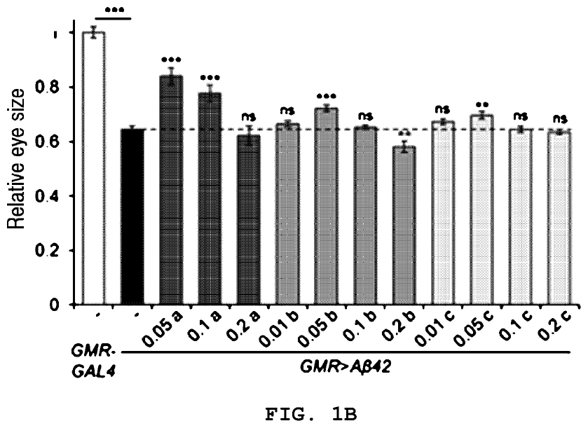

A phenotype in which the size of the eye is decreased was observed when overexpressing Aβ42 in the Drosophila eye. Using the same, Drosophila embryos for 0-6 hr in which Aβ42 was overexpressed specifically in the Drosophila eye using GMR-GAL4 were irradiated with a total of 0.01-0.2 Gy of low-dose radiation at different dose rates (¹³⁷Cs, 15.936 mGy/s, 3.147 mGy/s, 5 mGy/s), followed by incubation in an incubator at 25° C. until adulthood, and changes in the Alzheimer's disease phenotype in the Drosophila eye were compared. The symptom alleviation effects depending on the dose and dose rate of radiation are shown in FIGS. 1a and 1b.

With reference to FIGS. 1a and 1b, it was confirmed that the eye size of the Alzheimer's disease Drosophila model (GMR>Aβ42), which was decreased compared to normal Drosophila (GMR-GAL4), was changed due to low-dose radiation. When 0.05 Gy was applied at a dose rate of 15.936 mGy/s, the size of the eye increased the most. In particular, when a final dose of 0.05 Gy was applied, it can be seen that the size of the eye increased significantly at all three dose rates (a-c). In addition, the size of the eye increased even when 0.1 Gy was applied at 15.936 mGy/s. However, when 0.2 Gy was applied, the size of the eye decreased or was similar depending on the dose rate. Based on the above results, it was confirmed that low-dose radiation of 0.05 Gy (15.936 mGy/s) had the greatest effect on alleviating Alzheimer's disease.

### Example 2. Gene Group Specifically Responding to Low-Dose Radiation in Alzheimer's Disease Drosophila Model Depending on Function Thereof

Alzheimer's disease model (elav>Aβ42) Drosophila embryos irradiated with 0.05 Gy (15.936 mGy/s), exhibiting the best alleviation effect among the results investigated in Example 1, were incubated and raised in an incubator at 25° C. until adulthood, the head tissue of fully grown adult Drosophila was separated, and the entire gene expression change pattern thereof was investigated through an RNA sequencing method. In more detail, when the Alzheimer's disease Drosophila model was irradiated with radiation of 0.05 Gy at a dose rate of 15.936 mGy/s, genes the expression of which was doubled or more (increased expression: 82, decreased expression: 345, total genes: 427) were selected. Gene ontology analysis and KEGG pathway analysis were performed on the 427 genes thus obtained, and thus the genes were classified depending on the functions thereof.

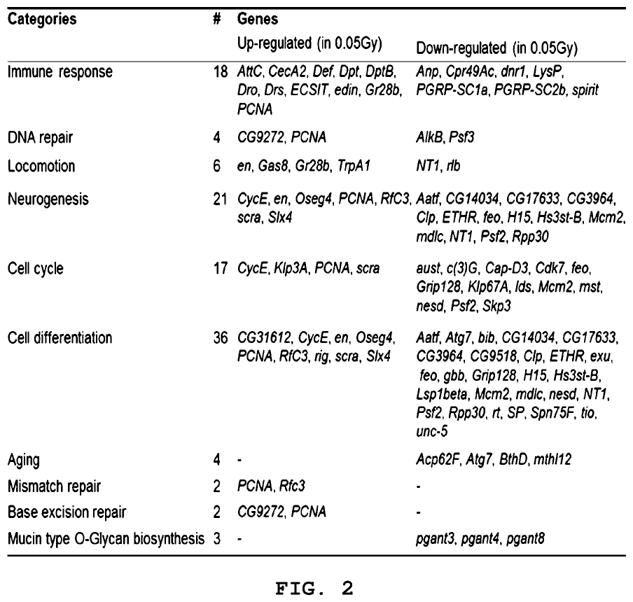

With reference to FIG. 2, in functional classifications of the genes changed due to low-dose radiation in the Alzheimer's disease Drosophila model, functional classifications thought to be related to Alzheimer's disease were selected, and genes belonging thereto were shown. Genes the expression of which was increased (up-regulated) and genes the expression of which was decreased (down-regulated) when radiation of 0.05 Gy was applied thereto were shown. The genes the expression of which was changed due to low-dose radiation were classified depending on the function thereof, and are shown in FIG. 2.

### Example 3. Gene Specifically Responding to Low-Dose Radiation Exhibiting Changes in Expression for Relieving Alzheimer's Disease

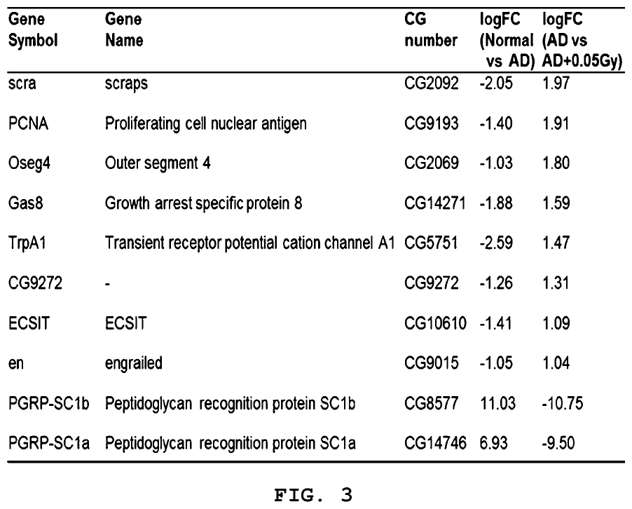

When compared with normal Drosophila among the genes classified and shown in Example 2, genes causing expression changes to relieve Alzheimer's disease symptoms through low-dose radiation of 0.05 Gy were selected. The genes thus selected have already been reported to be involved in immune response activation (PGRP-SC1a, PGRP-SC1b), neuronal regeneration (en, Oseg4, PCNA, scra), and motility function (Gas8, TrpA1). However, the present study suggested for the first time that the selected genes are associated with the effect of alleviating Alzheimer's disease by being specifically changed due to low-dose radiation. The selection results are shown in FIG. 3.

### Example 4. Changes in Expression Through Real-Time PCR of Representative Genes Exhibiting Changes in Expression for Relieving Alzheimer's Disease Upon Low-Dose Radiation

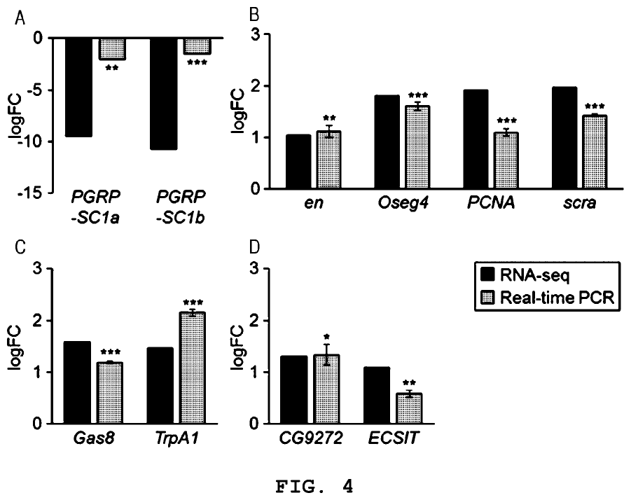

The changes in expression of genes that relieve Alzheimer's disease symptoms upon treatment with low-dose radiation shown in Example 3 were confirmed again based on mRNA level through real-time RT-PCR, whereby changes in gene expression due to low-dose radiation were verified. The results thereof are shown in FIG. 4.

Although specific embodiments of the present disclosure have been disclosed in detail as described above, it will be obvious to those skilled in the art that such description is merely of preferable exemplary embodiments, and is not to be construed to limit the scope of the present disclosure. Therefore, the substantial scope of the present disclosure will be defined by the appended claims and equivalents thereto.

Sequence Listing Free Text

Attach sequence listing in electronic form.

1. A biomarker for confirming treatment of a Drosophila degenerative disease, comprising:
a gene involved in immune response activation;
a gene involved in neuronal regeneration; and
a gene involved in motility function;
each of which is sensitive to low-dose radiation.

2. The biomarker of claim 1, wherein each of the genes is sensitive to the low-dose radiation at a cumulative dose of 0.01 to 0.2 Gy.

3. The biomarker of claim 1, wherein the gene involved in immune response activation comprises at least one selected from the group consisting of PGRP-SC1a and PGRP-SC1b.

4. The biomarker of claim 1, wherein the gene involved in neuronal regeneration comprises at least one selected from the group consisting of en, Oseg4, PCNA, and scra.

5. The biomarker of claim 1, wherein the gene involved in motility function comprises at least one selected from the group consisting of Gas8 and TrpA1.

6. The biomarker of claim 1, wherein the Drosophila degenerative disease is an Alzheimer's disease Drosophila model.

7. The biomarker of claim 6, wherein the Alzheimer's disease Drosophila model is GMR>Aβ42 or elav>Aβ42.

8. The biomarker of claim 1, wherein the degenerative disease is Alzheimer's disease.

9. The biomarker of claim 1, wherein the biomarker further comprises at least one gene selected from the group consisting of CG9272 and ECSIT.

10. The biomarker of claim 3, wherein the gene involved in neuronal regeneration comprises at least one selected from the group consisting of en, Oseg4, PCNA, and scra.

11. The biomarker of claim 3, wherein the gene involved in motility function comprises at least one selected from the group consisting of Gas8 and TrpA1.

12. The biomarker of claim 4, wherein the gene involved in motility function comprises at least one selected from the group consisting of Gas8 and TrpA1.

13. The biomarker of claim 10, wherein the gene involved in motility function comprises at least one selected from the group consisting of Gas8 and TrpA1.


SEQUENCE LISTING (30 sequences)

SEQ ID NO: 1 - DNA, 588 bp, Drosophila melanogaster
agcgatcgtcaactattacagctggtaatcatggtttccaaagtggctctcctcctcgccgtcctggtctgcagccagtacatggcccagggcgtctatgtcgtctccaaggcggagtggggtggtcgcggcgccaaatggaccgtaggcctgggcaactacctcagctacgccatcatccaccacaccgccggctcctactgcgagacccgtgcccagtgcaacgccgtgctgcagagcgtccagaactaccacatggactccctgggctggcccgacatcggctacaacttcctgatcggcggagacggcaacgtgtacgaggggcgtggctggaacaacatgggcgcccacgccgccgagtggaacccctacagcatcggcatcagcttcctgggcaactacaactgggacaccctggagccgaacatgatctccgccgcccagcagctgctcaacgacgccgtcaaccgtggccagctcagctccggctacatcctgtacggtcatcgccaggtcagcgccaccgaatgccccggcacccacatctggaacgagatccgcggctggtcccactggtctggctag

SEQ ID NO: 2 - DNA, 558 bp, Drosophila melanogaster
  CDS (1)..(555)
atggtttccaaagtggctctcctcctcgccgtcctggtctgcagccagtacatggcccagggcgtctatgtcgtctccaaggcggagtggggtggtcgcggcgccaaatggaccgtaggcctgggcaactacctcagctacgccatcatccaccacaccgccggctcctactgcgagacccgtgcccagtgcaacgccgtgctgcagagcgtccagaactaccacatggactccctgggctggcccgacatcggctacaacttcctgatcggcggagacggcaacgtgtacgaggggcgtggctggaacaacatgggcgcccacgccgccgagtggaacccctacagcatcggcatcagcttcctgggcaactacaactgggacaccctggagccgaacatgatctccgccgcccagcagctgctcaacgacgccgtcaaccgtggccagctcagctccggctacatcctgtacggtcatcgccaggtcagcgccaccgaatgccccggcacccacatctggaacgagatccgcggctggtcccactggtctggctag

SEQ ID NO: 3 - protein, 185 aa, Drosophila melanogaster
MVSKVALLLAVLVCSQYMAQGVYVVSKAEWGGRGAKWTVGLGNYLSYAIIHHTAGSYCETRAQCNAVLQSVQNYHMDSLGWPDIGYNFLIGGDGNVYEGRGWNNMGAHAAEWNPYSIGISFLGNYNWDTLEPNMISAAQQLLNDAVNRGQLSSGYILYGHRQVSATECPGTHIWNEIRGWSHWSG

SEQ ID NO: 4 - DNA, 1649 bp, Drosophila melanogaster
agcgatcgtcaactattacagctggtaatcatggtttccaaagtggctctcctcctcgccgtcctggtctgcagccagtatatggcccagggcgtctatgtcgtctccaaggcggagtggggtggtcgcggcgccaaatggaccgtaggcctgggcaactacctcagctacgccatcatccaccacaccgccggctcctactgcgagacccgtgcccagtgcaacgccgtgctgcagagcgtccagaactaccacatggactccctgggctggcccgacatcggctacaacttcctgatcggcggagacggcaacgtgtacgaggggcgtggctggaacaacatgggcgcccacgccgccgagtggaacccctacagcatcggcatcagcttcctgggcaactacaactgggacaccctggagccgaacatgatctccgccgcccagcagctgctcaacgacgccgtcaaccgtggccagctcagctccggctacatcctgtacggtcatcgccaggtcagcgccaccgaatgccccggcactcacatctggaacgagatccgcggctggtcccactggtctggttagagtggttcctgaaaaggttataaaatagtttgaatgatataatagcgaattctatattttcggggattcgggtattgatttgaatgcagtcgtttaaagggcaaagaaggggacatccaattgcaaagtcctgctcctattatccatctttgttttgcaactgaacgctcacgttcaactgtccaagtattcattccctcccttttactgaaacttttcaaatttataaaatcatgaggatgtgtgcctgctcagaaagttacagaacatcgtccgaactctttttttggaagtattttagttatttctttcatggaaagccaaagcgggtaatacaaaaatgtaaaattaactttcaaaaccggactgggatattaaataccgccaaaaccagcggtcaacttcacaatcggtagcagaccagagccaactatttgccacttgaaccgcagtctcattaacgacatcctcgttaacaaagttgctctgaatgggtttcagttgtttttgtgcttaataacttgcttaagctttcagtttttaagttcaactccgtcctgccataaggaaagcacttggtggcactcggttggttggacaaagtggattgacaaagtggttacagcccaacgcactcaccttgctgtccctctgcaggtctttcgagctgcacttctacttccgtgattacctgttacttatcaagatttacgcgctatgtctggtcattggaaagcgaaatttctgcaagtcagctaccttaacttgtggctgataagatgaactatagttctgtaatgcccccttttaatcagttgtttaataatttttgttataactaaatacgttatagtacccagctcagcaagcacaaaccattttaaataaataatttaacatattcatatacggtgggaaacatttcatacggacagccggatggatatacggacgggttgacagagggacgaaaatatggtcggaaacgctaccttctacctattaacatacctatcgataaatctattctaccccttttacataacctaaacttatattgt

SEQ ID NO: 5 - DNA, 558 bp, Drosophila melanogaster
  CDS (1)..(555)
atggtttccaaagtggctctcctcctcgccgtcctggtctgcagccagtatatggcccagggcgtctatgtcgtctccaaggcggagtggggtggtcgcggcgccaaatggaccgtaggcctgggcaactacctcagctacgccatcatccaccacaccgccggctcctactgcgagacccgtgcccagtgcaacgccgtgctgcagagcgtccagaactaccacatggactccctgggctggcccgacatcggctacaacttcctgatcggcggagacggcaacgtgtacgaggggcgtggctggaacaacatgggcgcccacgccgccgagtggaacccctacagcatcggcatcagcttcctgggcaactacaactgggacaccctggagccgaacatgatctccgccgcccagcagctgctcaacgacgccgtcaaccgtggccagctcagctccggctacatcctgtacggtcatcgccaggtcagcgccaccgaatgccccggcactcacatctggaacgagatccgcggctggtcccactggtctggttag

SEQ ID NO: 6 - protein, 185 aa, Drosophila melanogaster; identical to SEQ ID NO: 3

SEQ ID NO: 7 - DNA, 4205 bp, Drosophila melanogaster
gagggagcgagcgagagagcgctctggccagctaataggagtgagtgagccggcgaaaccggttcgcatggggcaggtgacaaggctaagagagagcgaaaatcgatcagtgtaaggccgaaaagacacatcttctactctcatctcttcatgtcactgtcaccaactgctgtcactcactcactcgctcgctcgcacacacttcgccagctacttgtgggatctcgccctctcgctcccgcactcggacctctctttccaccgtgacagttcaatcggctctcgcgttaactctccccgacgtcggcgctgcgattccgaagtagtcaactaattcagtcgttgcgctcgatgtgaacagacgtgcgtgtcggaacaacagttgcaaatcaaacacgaaagcataagccaaacaaaaaacaccaaacagagaagagaatcagaagattctgagcaatcagaagaatcagtggctcagtgtcaagtgacccagtgacaagtgtcttaagcgagttgcgatttagcaccaagtcgaaaccaatggccctggaggatcgctgcagtccacagtcagcgcccagccccattaccctacaaatgcagcatcttcaccaccagcaacagcagcagcagcaacagcagcagcaaatgcagcacctccaccagctgcagcaactgcagcagttgcaccaacagcaactggccgccggtgtcttccaccatccggcaatggccttcgatgccgctgcagccgccgctgctgcagctgctgctgcggccgcccacgctcatgctgctgcactgcagcagcgcctcagtggcagtggatcgcccgcatcctgctccacgcccgcctcgtccacgccgctgaccatcaaggaggaggaaagcgactccgtgatcggtgacatgagtttccacaatcagacgcacaccaccaacgaggaggaggaggcggaggaggatgacgacattgatgtggatgtggatgatacgtcggcgggcggacgcctgccaccacccgcccaccagcagcagtcgacggccaagccctcgctggccttttccatctccaacatcctgagcgatcgtttcggagatgtccagaagccgggcaagtcgatggagaaccaggccagcatattccgccccttcgaggcgagtcgttcccagactgccacgccctccgcctttacaagagtggatctgctggagtttagccggcaacagcaggctgccgccgcagccgctactgcggccatgatgctggaacgggccaacttccttaactgcttcaatccggctgcctatcccaggatacacgaggaaatcgtgcagagtcggctgcgcaggagtgcagccaatgccgtcatcccgccgcccatgagctccaagatgagcgatgccaatccagagaaatctgctctgggatccctgtgcaaggcggtctcgcagatcggacaacctgctgcccctacgatgacccaacctccgctgagtagcagtgccagcagcttggccagtccgccacccgcctccaatgcctcgaccattagcagcacctcttccgtggccaccagctcgagctcctcctcgtcgggttgctcctcggcggccagttccttgaactcctcgcccagtagccgactgggagccagtggatccggagtcaatgccagcagtccccagccgcagccaatcccgccgccatccgccgttagccgagattccggaatggagtcctcggatgacacgcgttccgagacgggatccaccaccacagagggcggcaagaacgagatgtggcccgcctgggtgtactgcacccgctacagcgatcgtcccagctcaggttagtatctatgcgattacctagcgtttaagtgggtcagaaagagggttcgtcgggcaccatcttcagtgttgtcaaaacaaggaagtcccaaaggttcgttaaaaaaaaaaagatgtgaaaatggtaaatggtatatggttacatggggaccacccttggggagcgttatgttggcggcagcctttcaagcagctctgttttgtgtcataaatccgaaggacactgccacaattaggcgacacgcggacaatggcaatgagcccgaatctgactcccatttctataaccatgcccattcccattcccatttcgtatccgaatccgaatccgattttgggcctcacgcagtgctgcgctgcttttgaagagcgacactttatgtgacaacatgtagcgcagagccagcgcttgtctatcggaactgttagatcctcaggtcgaaggtcaccgctcacgcacacacgccacgcagaagccgagaccctaaaacgcgctgacgattgacaggggaaaagtaggtgcgggggagatatgatggcgggggaagccccttcgaaaggagaagagttttccattgacaagcggagattttccttgcagctcttcttagatcgtgtgttaattgacgagttaagtgcttttttatggtcggcaatggttttttaatttggtgtcgatgcaatcgatggcagcccataagcgaggcgttgatggccccactccgcggctaggaattaatcaaacgatctccacttactggcggtcaattaaattataatttacaatgtgtggaaatctaataagcactacggattgctgcggtgccgcgttgccaattagccggtgacttggacttagtcgctaattagagagttggctagctggcaaatcatttgcctaaaatatgccgtagacctttgggttcgttaactcgccggagttctactcttctcttgcgctgacagctgacaaattgaacacttccccgccagcacgcgaaagggagctccagctccggccgaggaattccgcccagacatatgctaattgaatcattaatattattaatagtatgtggcgcatttatgcaaatagaactaacgtatttctcgtgtttttgtttattccaggaccccgctaccgccgccccaaacagccaaaggacaagaccaacgacgagaagcgtccacgcaccgcgttctccagcgagcagttggcccgccttaaggtgagattcagttcttttttccatatatggttatatggttataccaatatccatccatatccatatccaattgccctacgcttgttgtgtcatatttgcaccaaaatattgacaccgcccgctgctaatgcatcctggcaatgtggtgtcccgtccgtacttaaccaatcagccacgctcggccgaaaccgcaaataattgattttccctacctgactatacaacatggatatccttgccatccgaaactcatccagcattccatttcttccacttacagcgggagttcaacgagaatcgctatctgaccgagcggagacgccagcagctgagcagcgagttgggcctgaacgaggcgcagatcaagatctggttccagaacaagcgggccaagatcaagaagtcgacgggctccaaaaatccgctggcactgcagctgatggcccagggattgtacaaccacaccaccgtgccgctgaccaaggaggaggaggagctcgagatgcgcatgaacgggcagatcccctaagcgctgaccaatggttacccataaggggttactccttctgacgggggcgtgcaatattcgagggcgtacaaatggttttcatgtgatataattgtagcgtacatatgtttttgtatatatctctaaaaatatatatatatatacgtataatcctaacctagagtaagacccatccgtagcgaattcgagctgtaagttgttggcgtatttatttaaccacccctggatagccgaaagtattatcgtaatcacccgacacaaagcctatcgctatcgccgcacttcaaaagcttcgaccttcagacgtttattcctacacaaaacactatctatagttattactccctataaattacgcgcttgcaaccgctctgtaaattaagtaacttaacagttccctagcttaattcctagtttacacacttaaggattgctatgaaagagtattttagtttctaagcgaaatgttaacgagcaactaatagctggcaaatgcacctaagaaaataacaacgaaatgtttgtgctaaagcaagttcatgaaaagaaaattaaaaacaaaataaaagaattatggaaaaaaattaaaatgc

SEQ ID NO: 8 - DNA, 1659 bp, Drosophila melanogaster
  CDS (1)..(1656)
atggccctggaggatcgctgcagtccacagtcagcgcccagccccattaccctacaaatgcagcatcttcaccaccagcaacagcagcagcagcaacagcagcagcaaatgcagcacctccaccagctgcagcaactgcagcagttgcaccaacagcaactggccgccggtgtcttccaccatccggcaatggccttcgatgccgctgcagccgccgctgctgcagctgctgctgcggccgcccacgctcatgctgctgcactgcagcagcgcctcagtggcagtggatcgcccgcatcctgctccacgcccgcctcgtccacgccgctgaccatcaaggaggaggaaagcgactccgtgatcggtgacatgagtttccacaatcagacgcacaccaccaacgaggaggaggaggcggaggaggatgacgacattgatgtggatgtggatgatacgtcggcgggcggacgcctgccaccacccgcccaccagcagcagtcgacggccaagccctcgctggccttttccatctccaacatcctgagcgatcgtttcggagatgtccagaagccgggcaagtcgatggagaaccaggccagcatattccgccccttcgaggcgagtcgttcccagactgccacgccctccgcctttacaagagtggatctgctggagtttagccggcaacagcaggctgccgccgcagccgctactgcggccatgatgctggaacgggccaacttccttaactgcttcaatccggctgcctatcccaggatacacgaggaaatcgtgcagagtcggctgcgcaggagtgcagccaatgccgtcatcccgccgcccatgagctccaagatgagcgatgccaatccagagaaatctgctctgggatccctgtgcaaggcggtctcgcagatcggacaacctgctgcccctacgatgacccaacctccgctgagtagcagtgccagcagcttggccagtccgccacccgcctccaatgcctcgaccattagcagcacctcttccgtggccaccagctcgagctcctcctcgtcgggttgctcctcggcggccagttccttgaactcctcgcccagtagccgactgggagccagtggatccggagtcaatgccagcagtccccagccgcagccaatcccgccgccatccgccgttagccgagattccggaatggagtcctcggatgacacgcgttccgagacgggatccaccaccacagagggcggcaagaacgagatgtggcccgcctgggtgtactgcacccgctacagcgatcgtcccagctcaggaccccgctaccgccgccccaaacagccaaaggacaagaccaacgacgagaagcgtccacgcaccgcgttctccagcgagcagttggcccgccttaagcgggagttcaacgagaatcgctatctgaccgagcggagacgccagcagctgagcagcgagttgggcctgaacgaggcgcagatcaagatctggttccagaacaagcgggccaagatcaagaagtcgacgggctccaaaaatccgctggcactgcagctgatggcccagggattgtacaaccacaccaccgtgccgctgaccaaggaggaggaggagctcgagatgcgcatgaacgggcagatcccctaa

SEQ ID NO: 9 - protein, 552 aa, Drosophila melanogaster
MALEDRCSPQSAPSPITLQMQHLHHQQQQQQQQQQQMQHLHQLQQLQQLHQQQLAAGVFHHPAMAFDAAAAAAAAAAAAAAHAHAAALQQRLSGSGSPASCSTPASSTPLTIKEEESDSVIGDMSFHNQTHTTNEEEEAEEDDDIDVDVDDTSAGGRLPPPAHQQQSTAKPSLAFSISNILSDRFGDVQKPGKSMENQASIFRPFEASRSQTATPSAFTRVDLLEFSRQQQAAAAAATAAMMLERANFLNCFNPAAYPRIHEEIVQSRLRRSAANAVIPPPMSSKMSDANPEKSALGSLCKAVSQIGQPAAPTMTQPPLSSSASSLASPPPASNASTISSTSSVATSSSSSSSGCSSAASSLNSSPSSRLGASGSGVNASSPQPQPIPPPSAVSRDSGMESSDDTRSETGSTTTEGGKNEMWPAWVYCTRYSDRPSSGPRYRRPKQPKDKTNDEKRPRTAFSSEQLARLKREFNENRYLTERRRQQLSSELGLNEAQIKIWFQNKRAKIKKSTGSKNPLALQLMAQGLYNHTTVPLTKEEEELEMRMNGQIP

SEQ ID NO: 10 - DNA, 4775 bp, Drosophila melanogaster
ccacttgccaccaataccaattgaaccactttttgggcccaaaacaggcttgggtctttggccagctgacaaaccgaagttgggatcaccatggaaaccaattagttttgctgccacagcaaatgcggaaaaacattgaagagaactgaaagaatccaagtgcacttgcaactccaagatgttcgtttacctcagcaaaaaggtaggcttcacttagggtaccaacaaatggtctacaaggtctctcctccagattgccatacccaataatgtgaagctcaattgcatcgcttggaataaggaggagggctacatcgcagtggccggaacggatggactgcttaaggtcctgaaactggaccagggtgagtgccacgatcagggataggaagtaacctgggtcggacatctaggtactttctaatcttccttgtgcctaatcgaacgtgttgaatctcaattgctcaatactcattaaagcttactgtttccaataatcaagataaagaaagttaactacttccaagtatcaagatatatctcataacataacccgtttttaataccatatactgctccgatttgatcattactttatgcccatctccagccactcccaacggacagagcaagggcgggctggccgccgtttccaatttgtcgatgaaccagaccctggatgggcacaaggagtcggtgcgggtggtcacctggaacgatgcgcagcagaagctgacctcctcggacacggacggtgtgatcatggtgtggatgctgtacaagggatcctggtacgaggagatgaccaacgacagaaagaagtccacggtggccagcatgagctggacgtcggacggatcgcggatctgcatcgtctatgaggatggggcgattatagtgggctcagtggatggcaaccgcatctttggcaaggagctgaagggcacccatctgactggcgtgcagtggagtccggacaatcggctaatcctctttgcgctggccaacggggagtgtcatctgtacgataaccaaggcaactttgctgtaagttagtacttgattatccttcatacttacccactttggttggttatgtactccagatgaagctgcacatccagtgcgtgaacataagtggcggctcctcctccagaggtcaccgcatagccagcatctgctggttcagtggcagggtcgtccagagtcgaaagcgtccagttctggccatttgctatgagaacggaagggtgcagatcatgaggaacgaaaatgacgacggtaggaggtgatgcaagctctagagtctcctaactaacccacatgacttttagcacccgcaatcttcgacactgggatgcgcaatgtggacgccaagtggaaccatgatggcactgtgttggccatttgtggcaccaccttggacgcggtaagtccgacttcgcagcgcgacaccaaccaggtgtgcttctactcgccgctgggcaagatctaccgcactctgaaggtgcctggcaccgacatcacctcgcttagctgggagggcaaatcgctgcgcatcgccatggccgtggattcgttcatctacttcgccaacatccgaccggactacatctggtgttacttcgagaagactgtggtgttcctgaacagcggcagtgcaaccagggagtcgcccatgagcgtgatcaccttctggaacaccgtctcaaatcagagtttcctcaaggaggtggagcccaccttgtgcctggcgtccagcagcgagcattgcgtcctgggcgtcgagtgtgtcagcagcaacatcaaggagattgcgctgagcaccctggagaaccgaagtaatcctgcggacgatagggtctaccagttgctcctctgcaattcaattggcaccaccgtggattgtgagtagatcaccgttgttgatgtttcggactaatcccagttatttttagccaagtacacggatattagaccctgctttgtgggcatcaactcgagctatgtggccatagcgtcccaggaggagattctaatttggcactaccacacacccaaggtgggcactttcagtcatcaacctggttaccaattgactaacccccattgatctcccttcagagtgcctccaccctgcacaatgtgaaggcgcgcaaggagaagcggttccatatcgacgacacgcccacgggagtggaaatggccaaggacctgatgctgagcagtagcagtggcgatggtcactccacgcagcggggaatcagtgatcccatctgtgcgctggcactctccgagaagctgctccttgtcgccagggaatccggcgctatcaacgagtacagcatagcaaacgtggctctgaggaaccgccacctgatgaacgccaaggtctacaagatggctatcaactgcaattccacgtgggtagtccaccacactcccttgatactagtaatatttatactccttacttagacgagccgccattatcgaccatatgggcgtgatgaccctgctcgacctggacgacaacagggagacccaactgaacttcagtcgggtggagcgcaaggacgtttgggccgtgagctgggccacggataacccactgctgctggctctgatggagaaaacgcgcatgtatatcttccggggcaatgatcccgaggagcccgtgtcctgctccggctacatatgcaccttcgaggacttggagatcaccagtgtcctgctggacgacataatcagcgtgggtgagctgcagaatttttcgcatatcattcagctgagggtaaagtccctgcgcgataccgacgatctactggagcacgtgggactggaggatgccaagcagttcattgaggacaacccgcatccgaggctctggcgactgctggcggagtctgcgttgaagaaactggaactggagacggcggagaacgcctttgtccgttgtgcccactacccgggcattaagttggtcaagagactgcgcaccatacactccaaggagctgcagagggcggagatatcagccttctacggagagttcgaggaggcggagaagctctacctggacgcggatcgtcgcgatctggccatagagctgcgcatgaccctttgcgattggtttcgcgtggtgcagctgtacagaatgggaggatcgggagtgtcggaccagcagatggagatcgcttggcgggagattggccaccacttcgccaacctgcgctcctgggagagtgccagggaatactacgagaagtcccactacctggagggttacatggaggctctgtaccacttggagcagttcgacgacttggaaaagtgcgtggaacggctaccggagaagagcccgctgcttccgaagctggccgagatgctggcctccgtgggcatgtgctccgaggctgttcaagcccatcttcgattcggcgaccagaaggccgccgtcgccacctgcgttaatctccggcagtggggcgaggctgttgagctggcccagaggttccagctgccccaggtgcaaacgctcatagccaagcacgcggcacagttgctccaggagggacgcctcaaggaagccatcgagatgcagcggaatgctgggcgtcatctagatgcagctcgcctgctttctcaaatggcggaacgggagcaagagaagcgtgctccgctgctgagaatcaagaagctgtacgtgctggctgctctgctggccgaggagcacctcaaggctgtggccacgacggagatcgactacgccagcggcaggaacaccctactggactccattgccctggaggatgcggccgccatcgagaggctttggcactgcgccgaggcctaccacttcatgctcctagcccagaggcagcttcgcttcgggatcgtccacagtgcggtggtcactgcggtgaggctgcgcgactacgaggacgtcctgcctccagagcacatctacagcctgttggcactggccagctgtgcggatcgggccttcggaacctgctccaaggctttcatgaagttggagcaacaggctcatcttcccgaggctacccttcaacggtacgaggagttggcggctgggatcttcgccaagtacgatccggaggacaccactggcgatagggtggattgctattcctgcggagtgcccgtgccagataggtaagtgaagggtcgacgtcagaagctggattataatgatgatactcatggaatgccgatcctttcagctccccctcctgccccgagtgcaatgctcgcttcccggcgtgcatctcctccgggaaacccatcacccagccgacgaacaacatatggatctgtaccacctgccaccactgcgcagctcccaccgagatctccaggcaccgaacttgcccactgtgccacagtttgatcgtgtccatgacggtggagatctgaaatgagcggggactagtgttttaattactccagctgtttttcattctgcttatcccaggtggtacaggcgtatctactccgcaaataaacaatgtagttttaagatagaattcaaatttattttcaagtgaaaagtgttataagtggaaagagtgactgttcacagtgagtatttcctggggagtgcgctagggccacactactagctcatccaacactcgcaaagcggtcacactggcgagtgcagctaagtaagtcggttgtccgtttcttgctgaattttaaataagttaaataaatttctgcttcgtgcggtggtgtgcgtacgcaaatat

SEQ ID NO: 11 - DNA, 3618 bp, Drosophila melanogaster
  CDS (1)..(3615)
atgttcgtttacctcagcaaaaagattgccatacccaataatgtgaagctcaattgcatcgcttggaataaggaggagggctacatcgcagtggccggaacggatggactgcttaaggtcctgaaactggaccaggccactcccaacggacagagcaagggcgggctggccgccgtttccaatttgtcgatgaaccagaccctggatgggcacaaggagtcggtgcgggtggtcacctggaacgatgcgcagcagaagctgacctcctcggacacggacggtgtgatcatggtgtggatgctgtacaagggatcctggtacgaggagatgaccaacgacagaaagaagtccacggtggccagcatgagctggacgtcggacggatcgcggatctgcatcgtctatgaggatggggcgattatagtgggctcagtggatggcaaccgcatctttggcaaggagctgaagggcacccatctgactggcgtgcagtggagtccggacaatcggctaatcctctttgcgctggccaacggggagtgtcatctgtacgataaccaaggcaactttgctatgaagctgcacatccagtgcgtgaacataagtggcggctcctcctccagaggtcaccgcatagccagcatctgctggttcagtggcagggtcgtccagagtcgaaagcgtccagttctggccatttgctatgagaacggaagggtgcagatcatgaggaacgaaaatgacgacgcacccgcaatcttcgacactgggatgcgcaatgtggacgccaagtggaaccatgatggcactgtgttggccatttgtggcaccaccttggacgcggtaagtccgacttcgcagcgcgacaccaaccaggtgtgcttctactcgccgctgggcaagatctaccgcactctgaaggtgcctggcaccgacatcacctcgcttagctgggagggcaaatcgctgcgcatcgccatggccgtggattcgttcatctacttcgccaacatccgaccggactacatctggtgttacttcgagaagactgtggtgttcctgaacagcggcagtgcaaccagggagtcgcccatgagcgtgatcaccttctggaacaccgtctcaaatcagagtttcctcaaggaggtggagcccaccttgtgcctggcgtccagcagcgagcattgcgtcctgggcgtcgagtgtgtcagcagcaacatcaaggagattgcgctgagcaccctggagaaccgaagtaatcctgcggacgatagggtctaccagttgctcctctgcaattcaattggcaccaccgtggattccaagtacacggatattagaccctgctttgtgggcatcaactcgagctatgtggccatagcgtcccaggaggagattctaatttggcactaccacacacccaagagtgcctccaccctgcacaatgtgaaggcgcgcaaggagaagcggttccatatcgacgacacgcccacgggagtggaaatggccaaggacctgatgctgagcagtagcagtggcgatggtcactccacgcagcggggaatcagtgatcccatctgtgcgctggcactctccgagaagctgctccttgtcgccagggaatccggcgctatcaacgagtacagcatagcaaacgtggctctgaggaaccgccacctgatgaacgccaaggtctacaagatggctatcaactgcaattccacacgagccgccattatcgaccatatgggcgtgatgaccctgctcgacctggacgacaacagggagacccaactgaacttcagtcgggtggagcgcaaggacgtttgggccgtgagctgggccacggataacccactgctgctggctctgatggagaaaacgcgcatgtatatcttccggggcaatgatcccgaggagcccgtgtcctgctccggctacatatgcaccttcgaggacttggagatcaccagtgtcctgctggacgacataatcagcgtgggtgagctgcagaatttttcgcatatcattcagctgagggtaaagtccctgcgcgataccgacgatctactggagcacgtgggactggaggatgccaagcagttcattgaggacaacccgcatccgaggctctggcgactgctggcggagtctgcgttgaagaaactggaactggagacggcggagaacgcctttgtccgttgtgcccactacccgggcattaagttggtcaagagactgcgcaccatacactccaaggagctgcagagggcggagatatcagccttctacggagagttcgaggaggcggagaagctctacctggacgcggatcgtcgcgatctggccatagagctgcgcatgaccctttgcgattggtttcgcgtggtgcagctgtacagaatgggaggatcgggagtgtcggaccagcagatggagatcgcttggcgggagattggccaccacttcgccaacctgcgctcctgggagagtgccagggaatactacgagaagtcccactacctggagggttacatggaggctctgtaccacttggagcagttcgacgacttggaaaagtgcgtggaacggctaccggagaagagcccgctgcttccgaagctggccgagatgctggcctccgtgggcatgtgctccgaggctgttcaagcccatcttcgattcggcgaccagaaggccgccgtcgccacctgcgttaatctccggcagtggggcgaggctgttgagctggcccagaggttccagctgccccaggtgcaaacgctcatagccaagcacgcggcacagttgctccaggagggacgcctcaaggaagccatcgagatgcagcggaatgctgggcgtcatctagatgcagctcgcctgctttctcaaatggcggaacgggagcaagagaagcgtgctccgctgctgagaatcaagaagctgtacgtgctggctgctctgctggccgaggagcacctcaaggctgtggccacgacggagatcgactacgccagcggcaggaacaccctactggactccattgccctggaggatgcggccgccatcgagaggctttggcactgcgccgaggcctaccacttcatgctcctagcccagaggcagcttcgcttcgggatcgtccacagtgcggtggtcactgcggtgaggctgcgcgactacgaggacgtcctgcctccagagcacatctacagcctgttggcactggccagctgtgcggatcgggccttcggaacctgctccaaggctttcatgaagttggagcaacaggctcatcttcccgaggctacccttcaacggtacgaggagttggcggctgggatcttcgccaagtacgatccggaggacaccactggcgatagggtggattgctattcctgcggagtgcccgtgccagatagctccccctcctgccccgagtgcaatgctcgcttcccggcgtgcatctcctccgggaaacccatcacccagccgacgaacaacatatggatctgtaccacctgccaccactgcgcagctcccaccgagatctccaggcaccgaacttgcccactgtgccacagtttgatcgtgtccatgacggtggagatctga

SEQ ID NO: 12 - protein, 1205 aa, Drosophila melanogaster
MFVYLSKKIAIPNNVKLNCIAWNKEEGYIAVAGTDGLLKVLKLDQATPNGQSKGGLAAVSNLSMNQTLDGHKESVRVVTWNDAQQKLTSSDTDGVIMVWMLYKGSWYEEMTNDRKKSTVASMSWTSDGSRICIVYEDGAIIVGSVDGNRIFGKELKGTHLTGVQWSPDNRLILFALANGECHLYDNQGNFAMKLHIQCVNISGGSSSRGHRIASICWFSGRVVQSRKRPVLAICYENGRVQIMRNENDDAPAIFDTGMRNVDAKWNHDGTVLAICGTTLDAVSPTSQRDTNQVCFYSPLGKIYRTLKVPGTDITSLSWEGKSLRIAMAVDSFIYFANIRPDYIWCYFEKTVVFLNSGSATRESPMSVITFWNTVSNQSFLKEVEPTLCLASSSEHCVLGVECVSSNIKEIALSTLENRSNPADDRVYQLLLCNSIGTTVDSKYTDIRPCFVGINSSYVAIASQEEILIWHYHTPKSASTLHNVKARKEKRFHIDDTPTGVEMAKDLMLSSSSGDGHSTQRGISDPICALALSEKLLLVARESGAINEYSIANVALRNRHLMNAKVYKMAINCNSTRAAIIDHMGVMTLLDLDDNRETQLNFSRVERKDVWAVSWATDNPLLLALMEKTRMYIFRGNDPEEPVSCSGYICTFEDLEITSVLLDDIISVGELQNFSHIIQLRVKSLRDTDDLLEHVGLEDAKQFIEDNPHPRLWRLLAESALKKLELETAENAFVRCAHYPGIKLVKRLRTIHSKELQRAEISAFYGEFEEAEKLYLDADRRDLAIELRMTLCDWFRVVQLYRMGGSGVSDQQMEIAWREIGHHFANLRSWESAREYYEKSHYLEGYMEALYHLEQFDDLEKCVERLPEKSPLLPKLAEMLASVGMCSEAVQAHLRFGDQKAAVATCVNLRQWGEAVELAQRFQLPQVQTLIAKHAAQLLQEGRLKEAIEMQRNAGRHLDAARLLSQMAEREQEKRAPLLRIKKLYVLAALLAEEHLKAVATTEIDYASGRNTLLDSIALEDAAAIERLWHCAEAYHFMLLAQRQLRFGIVHSAVVTAVRLRDYEDVLPPEHIYSLLALASCADRAFGTCSKAFMKLEQQAHLPEATLQRYEELAAGIFAKYDPEDTTGDRVDCYSCGVPVPDSSPSCPECNARFPACISSGKPITQPTNNIWICTTCHHCAAPTEISRHRTCPLCHSLIVSMTVEI

SEQ ID NO: 13 - DNA, 1130 bp, Drosophila melanogaster
gtggcttttcacatccctatcccgctcatttagcccgcctgaaagtaaaaaaaaaaaacagcccacgttaatcattcatcccaaagtcacagccgcggtaacattactgctgttaaattcttaagcccgtcatcagtatttaaataataaaacacattcaatatgttcgaggcacgcctgggtcaagccaccatcctgaagaagatcttggatgccatcaaggatctgctcaatgaggcaaccttcgattgcagcgactccggcattcaggtaaaaattgcacaaaaaagattttaaatgcatatccctaacaccttgttcaacttacagctacaggccatggacaactcccatgtgtcgcttgtctcgctgaccctgcgttccgatggcttcgacaagtttcgctgcgaccgcaatctctccatgggcatgaatctgggcagcatggccaagattctgaaatgcgccaacaacgaggacaatgtgacgatgaaggcgcaggataacgccgacactgtcaccatcatgttcgaatcggctaaccaggagaaggtatcggactacgagatgaaactgatgaacctcgaccaggagcacctgggcataccggagacagacttctcgtgcgtggtccgcatgccggccatggagttcgctcgcatctgccgcgatctggcgcagttcagcgaatccgttgtgatctgctgcaccaaggagggcgtcaagttctcggccagcggcgatgtgggcaccgccaacattaagctagcccaaaccggctctgtcgacaaggaggaggaggcggtgatcatcgagatgcaggagccggtgacgctgacatttgcctgtcgctacctgaacgccttcacaaaggcgacgccattgtccacccaagtgcagctgtcgatgtgcgcagatgttccgctggtagtcgagtatgcgatcaaggatctgggtcacattcgctactacctggcacccaagatcgaggacaacgagacataagtcagctgtgttcctcatatttatgtccccgcatcgtcaccactcatcttcccacgttcacttcattcctaacttttaagtaaatccgcattttttgatcaataaaagctatactgtgtaatgtt

SEQ ID NO: 14 - DNA, 783 bp, Drosophila melanogaster
  CDS (1)..(780)
atgttcgaggcacgcctgggtcaagccaccatcctgaagaagatcttggatgccatcaaggatctgctcaatgaggcaaccttcgattgcagcgactccggcattcagctacaggccatggacaactcccatgtgtcgcttgtctcgctgaccctgcgttccgatggcttcgacaagtttcgctgcgaccgcaatctctccatgggcatgaatctgggcagcatggccaagattctgaaatgcgccaacaacgaggacaatgtgacgatgaaggcgcaggataacgccgacactgtcaccatcatgttcgaatcggctaaccaggagaaggtatcggactacgagatgaaactgatgaacctcgaccaggagcacctgggcataccggagacagacttctcgtgcgtggtccgcatgccggccatggagttcgctcgcatctgccgcgatctggcgcagttcagcgaatccgttgtgatctgctgcaccaaggagggcgtcaagttctcggccagcggcgatgtgggcaccgccaacattaagctagcccaaaccggctctgtcgacaaggaggaggaggcggtgatcatcgagatgcaggagccggtgacgctgacatttgcctgtcgctacctgaacgccttcacaaaggcgacgccattgtccacccaagtgcagctgtcgatgtgcgcagatgttccgctggtagtcgagtatgcgatcaaggatctgggtcacattcgctactacctggcacccaagatcgaggacaacgagacataa

SEQ ID NO: 15 - protein, 260 aa, Drosophila melanogaster
MFEARLGQATILKKILDAIKDLLNEATFDCSDSGIQLQAMDNSHVSLVSLTLRSDGFDKFRCDRNLSMGMNLGSMAKILKCANNEDNVTMKAQDNADTVTIMFESANQEKVSDYEMKLMNLDQEHLGIPETDFSCVVRMPAMEFARICRDLAQFSESVVICCTKEGVKFSASGDVGTANIKLAQTGSVDKEEEAVIIEMQEPVTLTFACRYLNAFTKATPLSTQVQLSMCADVPLVVEYAIKDLGHIRYYLAPKIEDNET

SEQ ID NO: 16 - DNA, 5124 bp, Drosophila melanogaster
ccaccctgccaaatacgacaaagtggcagcccttggcaactcaccactggcattttctccccaatttcggtatttcgtcgtggtcagttgagggtcctatggcatttgcgcgtcgttttgaaattattcaacacatctgaaaagccaaccgccgaaacgagcagaagcgacgcgcagaatttcgaatgcaaacggacgcgcaacccgccagtgaaaaacaaacatcaaatatcgccaacgtggagtgtcgagcagtacagtgaattcaatagcatagtgcgcgcaatcacaactcgcatttgcatttgcagctgcagcaacaacaatggacccgtttactcaggtacgaggccaattcgcacacagcaaagccctttgtctggtgattgcactggtgttagtgaaggcacccgcggattttcactgctatttcccattttcccttgcagcacatgctcgagaaggcggaacagcgcagtcgcgcccttggcatcagcaatgccagcaaatttccgctcgtcgagtgcagcgttccaagctcctccgccacctccgcatccggcggggatgctggggtcctggcaccgaggagccggtcgcctggaggccagagcgcggctagcggcggcggcaaggtcgttacgttgggaaaggccacgctggaggcgtcgccggcgaagccgctgcgtcactatacggcggtaaacaaggaaaacttggatatgggcatagagataaacataaccacggacaagccaattggggtaagttttcccgttcttttctgggttcttcccttttgctcatccttgctgcactaccttcaggtgcaagttgagattcaggagcaggaggtgacagatgacgaagagcaggcggaaggaggtgcgctaaatcccctgctggaggcggaaccagtaaaccagcccctagccaggctaagggacacttcccgcagccgactgcaacgcatgggtgccctgtactcaaacacggacgatctatcctccccaatccaccgaaccgaggggcagttccatgtgacaacgggtgaggaagaggactgcggcaaccgtagcagcaggcaaccaaaacaacggttgggcaaactggccgccttggcggataccatcaatcagtgggaggatgacacgtcgcaccacgaagtgcacagactgctcgaagcacctccaccgaaaccgcatttgtccagtcgaagggccgagaagggtccagctccactgccacccaagaaggatgaagtcgacgaggcaagcaggaccaagcagctgaagtgggatcccaaggtattgagctctctggaggcacaaggttttcagcgccgcgaatcatcgaccattaaacacacatatgattatgccaaacaggaagaggcggccccagcctccaaagtggaagatgcggtattaactgccaagccgccagtgccgcagaaatcaacgacggtcagccaggtcgccaagaactttgcatcttccgccccagcgcctaagcccgcaccagcgcctgctgttagtgtgaagtccggcttggtttccggacgggcggctctctttgaaaacaagggaaccggaggacagtcgcaaggcctacgcaaccagaaggatccttgtgagctgtcgctgaaggagcggatgaagctgttcgaaacgggcaacaacaaagcgatgttgccgatggcgccaataggatctgctcccagtattacccaaatccgagcggaagaggtgaaacgtgagtttggcagcccttctatattcataatagctactcatactcattttttttttgttaacagaacatttagctgcaatgcatccggtgactgctgccgcagctactaccgtggttgcagcaaccaagccgaaacaggaaaacaagctgcgtgacaaggtggccgctttggtggcaaatgcgcaatcaagtgcagagacgcgtatcaaggacattgatcgccaaaggcaggaagacatgcagattatttctaatcgctttaacaaacaaaaggagctctttgacaatcaaccatccgatagttctgtggctgctcaagctcgacctcccgcgccagctcccagcagagtagtgcggcccatgccaccgcctccaccaccgcccatcgctgctctctcgcccggactggccagcagcaagaggcgatcacgtaagaaatgactaaaccctttatctaaggacttgtccttatacatccgcaatttcagctggtgatgcgcccaccactgacgaggattcgaagcgagcccgcaaatcgcattcggatcgtctgtatcccgccctgtccgacctcgactccagcggtgacaactgctgcgccgccgaaactgcctccgctacggatgacagccatcaacaggacgaggaggaaaccgaaaggtgcgctgaaaaatgctattcctgcttgctatttctgctctgcatcgagtagcattccaagactgataggcctaagagacatagctttaaaccccatcccctagacaagaccaacaataaaatcgctttcttaactaaccgtttatctttgattcgtgcagttgcatggatgagtccgacgaccagtcacagactgaggacagtagcgccggcatgtgcaatggcagtcttggccgcgagataatgagcgcagtgcagcgcaacgaagtggagatgcagcagcagcagacgggcaagaaggtaagtgcaggtaaaaactgtcaacacagcgggtggtcaccattaacacgatttcacacctttccagactgtgcgctatgcggaccaggacatgtattacgatgacagctctctgaactcgtcgcaggtctcagcgggcatcgatgattatctggatgaagcacttgtcgaggactacggcagcactcaggatgaccagagcgacagcggggacgagcaaaatgccagtcggctgtccttgggcgtgagtacttgctgattaagtttcctcgtatatcaaaacaattctttaattacattttcagagcaaaggaacgacggcctcaaacagcttttccttccgaaaaaatcctgcctctatttgcacgcctatcgaagagcaccatgagatggagatggacctgcagacgccactgctcagtggtgcccagccggtcaagtctgaattgagtgtcaaccaggacaacgacaacctggttaccctggtgcacacggtcagcttctatcgccgccaacagagcgctaacgtgagttgattacttatatatcacaactaacaaattctaagtactcgttgtttatagagctccaattccacaccggttcgcaagatctgtcgagagcagcaggtcatgcgatcagccctagcaggcgattgtcatgccaagcacagactggagtacgactctcctcagcagtctgattatgtcgcagcagcaactgacatagctgatcagaccgacgaggacgatgaagagatgcagaatgcgcgggaagtaaacgatgcatcgcaggcacaagacaagatcaaaaagctgttgagcgaggtgtgcaagcagcagcaggtgataggacaagccagtcaggccctcaacctttgtgctgccaccgtagaattctccggctccacagagtccgtggagggagagcgatatctccttcttgcaagtaagtgtgatcttcagtgtattcaagtataaattgcctgttcgttgaattccaaaccgaattggcaatttgaatgtccaattaagctgaacatctctttaatatccttgcagcccatcgacgccaggcgtgtttggatgaagtccagcgtttaagagttgagaacagtattcgtccggtgggtgcaccaaaagaaaagggcctactgacggtcaaggacataaccattccactgcgacaggagtacgtgcgtaaaatggcctcgaacaacattaacggccatcatcttgtgtgcctcctgaagtacaatgagcacgtgctggccaccaagacagtacccacgatgccagggctgctgtctgtcaagtttcccgatgtcctgcaactaaacaatgtatatgctgacttcagggtaaatattgctttttgcttctttagccaatattcgattttattgcgaaaacttcgtctgcagatcacgctagaaatctatggcatgttggcccaacgcgatcagctgcctcacgagctgaaataccacatcaacttgaacaagaagggtggcatcaagacgccaaagaagaagggcggcgagaatcgactggtgatgccgccagtacaaagtccggcgggaccgcatgtggtacggacaccgcagctggtgcaatacggctttgccattttctcgctgcgtgaaattcaacgcaccacatggacgctgacccaggtgctgggcgtcagtccactggagggtgtggttcacatgaaagtcaattgcgaactatccgtaagcgtggagtacaagggattccttaccatgttcgaagacatctccggtttcggggcatggcatcgtcgttggtgctatctgaatggctccgtgatcaactactggaagtacccggatgatgagaagcgcaagacgccaatgggtagtatagatctgaattcctgcacttcgcagaaggtgaccacggcaccgcgcgacatttgcgcccgtctcaacaccatgctgctggagtgcgaacggccggcgctggagacagaccaagagtccttgataatcgtacccaacggacgtaccaccacggtgcgccatctgctctcagccgacacaaaggaggagcgcgaggagtggtgcgcctaccttaacaaggcactgacactgctgcgcgcctggggaaccacccactgacccgctgacccattgcaattttgtcagcagcacggacaatgaccacaaacgggcgggttgttaacagttttctctttttaagttactcaatttgctattttaatcgtgttcgcattggtattaattttgttttatgtttccaattcaaatttttcgtttccatcttggagtgggacacgttcgtgttcgtctatatcgacataactttttacttatgtacatttaatttaggatgactcgctaaatatatatgtgtaaacccccgt

SEQ ID NO: 17 - DNA, 3639 bp, Drosophila melanogaster
  CDS (1)..(3636)
atggacccgtttactcagcacatgctcgagaaggcggaacagcgcagtcgcgcccttggcatcagcaatgccagcaaatttccgctcgtcgagtgcagcgttccaagctcctccgccacctccgcatccggcggggatgctggggtcctggcaccgaggagccggtcgcctggaggccagagcgcggctagcggcggcggcaaggtcgttacgttgggaaaggccacgctggaggcgtcgccggcgaagccgctgcgtcactatacggcggtaaacaaggaaaacttggatatgggcatagagataaacataaccacggacaagccaattggggtgcaagttgagattcaggagcaggaggtgacagatgacgaagagcaggcggaaggaggtgcgctaaatcccctgctggaggcggaaccagtaaaccagcccctagccaggctaagggacacttcccgcagccgactgcaacgcatgggtgccctgtactcaaacacggacgatctatcctccccaatccaccgaaccgaggggcagttccatgtgacaacgggtgaggaagaggactgcggcaaccgtagcagcaggcaaccaaaacaacggttgggcaaactggccgccttggcggataccatcaatcagtgggaggatgacacgtcgcaccacgaagtgcacagactgctcgaagcacctccaccgaaaccgcatttgtccagtcgaagggccgagaagggtccagctccactgccacccaagaaggatgaagtcgacgaggcaagcaggaccaagcagctgaagtgggatcccaaggaagaggcggccccagcctccaaagtggaagatgcggtattaactgccaagccgccagtgccgcagaaatcaacgacggtcagccaggtcgccaagaactttgcatcttccgccccagcgcctaagcccgcaccagcgcctgctgttagtgtgaagtccggcttggtttccggacgggcggctctctttgaaaacaagggaaccggaggacagtcgcaaggcctacgcaaccagaaggatccttgtgagctgtcgctgaaggagcggatgaagctgttcgaaacgggcaacaacaaagcgatgttgccgatggcgccaataggatctgctcccagtattacccaaatccgagcggaagaggtgaaacaacatttagctgcaatgcatccggtgactgctgccgcagctactaccgtggttgcagcaaccaagccgaaacaggaaaacaagctgcgtgacaaggtggccgctttggtggcaaatgcgcaatcaagtgcagagacgcgtatcaaggacattgatcgccaaaggcaggaagacatgcagattatttctaatcgctttaacaaacaaaaggagctctttgacaatcaaccatccgatagttctgtggctgctcaagctcgacctcccgcgccagctcccagcagagtagtgcggcccatgccaccgcctccaccaccgcccatcgctgctctctcgcccggactggccagcagcaagaggcgatcacctggtgatgcgcccaccactgacgaggattcgaagcgagcccgcaaatcgcattcggatcgtctgtatcccgccctgtccgacctcgactccagcggtgacaactgctgcgccgccgaaactgcctccgctacggatgacagccatcaacaggacgaggaggaaaccgaaagttgcatggatgagtccgacgaccagtcacagactgaggacagtagcgccggcatgtgcaatggcagtcttggccgcgagataatgagcgcagtgcagcgcaacgaagtggagatgcagcagcagcagacgggcaagaagactgtgcgctatgcggaccaggacatgtattacgatgacagctctctgaactcgtcgcaggtctcagcgggcatcgatgattatctggatgaagcacttgtcgaggactacggcagcactcaggatgaccagagcgacagcggggacgagcaaaatgccagtcggctgtccttgggcagcaaaggaacgacggcctcaaacagcttttccttccgaaaaaatcctgcctctatttgcacgcctatcgaagagcaccatgagatggagatggacctgcagacgccactgctcagtggtgcccagccggtcaagtctgaattgagtgtcaaccaggacaacgacaacctggttaccctggtgcacacggtcagcttctatcgccgccaacagagcgctaacagctccaattccacaccggttcgcaagatctgtcgagagcagcaggtcatgcgatcagccctagcaggcgattgtcatgccaagcacagactggagtacgactctcctcagcagtctgattatgtcgcagcagcaactgacatagctgatcagaccgacgaggacgatgaagagatgcagaatgcgcgggaagtaaacgatgcatcgcaggcacaagacaagatcaaaaagctgttgagcgaggtgtgcaagcagcagcaggtgataggacaagccagtcaggccctcaacctttgtgctgccaccgtagaattctccggctccacagagtccgtggagggagagcgatatctccttcttgcaacccatcgacgccaggcgtgtttggatgaagtccagcgtttaagagttgagaacagtattcgtccggtgggtgcaccaaaagaaaagggcctactgacggtcaaggacataaccattccactgcgacaggagtacgtgcgtaaaatggcctcgaacaacattaacggccatcatcttgtgtgcctcctgaagtacaatgagcacgtgctggccaccaagacagtacccacgatgccagggctgctgtctgtcaagtttcccgatgtcctgcaactaaacaatgtatatgctgacttcaggatcacgctagaaatctatggcatgttggcccaacgcgatcagctgcctcacgagctgaaataccacatcaacttgaacaagaagggtggcatcaagacgccaaagaagaagggcggcgagaatcgactggtgatgccgccagtacaaagtccggcgggaccgcatgtggtacggacaccgcagctggtgcaatacggctttgccattttctcgctgcgtgaaattcaacgcaccacatggacgctgacccaggtgctgggcgtcagtccactggagggtgtggttcacatgaaagtcaattgcgaactatccgtaagcgtggagtacaagggattccttaccatgttcgaagacatctccggtttcggggcatggcatcgtcgttggtgctatctgaatggctccgtgatcaactactggaagtacccggatgatgagaagcgcaagacgccaatgggtagtatagatctgaattcctgcacttcgcagaaggtgaccacggcaccgcgcgacatttgcgcccgtctcaacaccatgctgctggagtgcgaacggccggcgctggagacagaccaagagtccttgataatcgtacccaacggacgtaccaccacggtgcgccatctgctctcagccgacacaaaggaggagcgcgaggagtggtgcgcctaccttaacaaggcactgacactgctgcgcgcctggggaaccacccactga

SEQ ID NO: 18 - protein, 1212 aa, Drosophila melanogaster
MDPFTQHMLEKAEQRSRALGISNASKFPLVECSVPSSSATSASGGDAGVLAPRSRSPGGQSAASGGGKVVTLGKATLEASPAKPLRHYTAVNKENLDMGIEINITTDKPIGVQVEIQEQEVTDDEEQAEGGALNPLLEAEPVNQPLARLRDTSRSRLQRMGALYSNTDDLSSPIHRTEGQFHVTTGEEEDCGNRSSRQPKQRLGKLAALADTINQWEDDTSHHEVHRLLEAPPPKPHLSSRRAEKGPAPLPPKKDEVDEASRTKQLKWDPKEEAAPASKVEDAVLTAKPPVPQKSTTVSQVAKNFASSAPAPKPAPAPAVSVKSGLVSGRAALFENKGTGGQSQGLRNQKDPCELSLKERMKLFETGNNKAMLPMAPIGSAPSITQIRAEEVKQHLAAMHPVTAAAATTVVAATKPKQENKLRDKVAALVANAQSSAETRIKDIDRQRQEDMQIISNRFNKQKELFDNQPSDSSVAAQARPPAPAPSRVVRPMPPPPPPPIAALSPGLASSKRRSPGDAPTTDEDSKRARKSHSDRLYPALSDLDSSGDNCCAAETASATDDSHQQDEEETESCMDESDDQSQTEDSSAGMCNGSLGREIMSAVQRNEVEMQQQQTGKKTVRYADQDMYYDDSSLNSSQVSAGIDDYLDEALVEDYGSTQDDQSDSGDEQNASRLSLGSKGTTASNSFSFRKNPASICTPIEEHHEMEMDLQTPLLSGAQPVKSELSVNQDNDNLVTLVHTVSFYRRQQSANSSNSTPVRKICREQQVMRSALAGDCHAKHRLEYDSPQQSDYVAAATDIADQTDEDDEEMQNAREVNDASQAQDKIKKLLSEVCKQQQVIGQASQALNLCAATVEFSGSTESVEGERYLLLATHRRQACLDEVQRLRVENSIRPVGAPKEKGLLTVKDITIPLRQEYVRKMASNNINGHHLVCLLKYNEHVLATKTVPTMPGLLSVKFPDVLQLNNVYADFRITLEIYGMLAQRDQLPHELKYHINLNKKGGIKTPKKKGGENRLVMPPVQSPAGPHVVRTPQLVQYGFAIFSLREIQRTTWTLTQVLGVSPLEGVVHMKVNCELSVSVEYKGFLTMFEDISGFGAWHRRWCYLNGSVINYWKYPDDEKRKTPMGSIDLNSCTSQKVTTAPRDICARLNTMLLECERPALETDQESLIIVPNGRTTTVRHLLSADTKEEREEWCAYLNKALTLLRAWGTTH

SEQ ID NO: 19 - DNA, 13841 bp, Drosophila melanogaster
caaattttgaattttgccagttcaaaaccatttgtccctgccgtagaaatataattgagagaagactgaaccgaaaaaacaacgcttcaacctttaacctcttctttcggttgcccggagacacacacacacacacacacgaatacacgagacacccacaacacacacattggcaactgctttatcgataagtttgtttggttgttggaccgtagaacgccaatagctcagttggcaggaaagaccaagccgaagtggttcttattcttagcagaggaatcgaagcagaggaagaagcaccggcagcagtatggtgagcaataagttgttaaatcacattaacaaagcagtgcacccattcggccgccttatggccgcactgcttggccagcagcagtttccgcctctatccgatttccggttccatgccgctgctccttttggtctcagcttttcagattttttttttcagttgcactgcaagcggcgcattcaatttttcatgccaagccaggctgttgctgttgctgcatatacaaactgcactttgcgagtgggctacagtgaagcctcggcaaggcgctgtcctgctactgctagtcaaaatgtcaacgttggtatggcgcattcatttcaaagcaactttagtagtcgttctgtctatataagaacatatgtagggagaacctaagttagtataactattccctttacagtggctccactgtgtatttatagcagtcaagcaggagtcctcctctccaactcctgctggttgtctattttcgatcatttgtttatgtatagacctgcccccaacatatatccgccttttttccatcggctacatatatatgtttatatttatatttatatttatatatatatatatataaatatatacattgcgcctttggacatgcgaaagcgcggttggcgacgggcgcaggcggcttcgaaaatttttgaataatttgcagtcataaaatatgcaataagccagcagccaacagccaacagctgttgcgaaattgttttgaaagccgcgccatcacattatttaaagcaacgatggcagttgcccgctttctccgcctttcgctgcctttctcccacgtgaagggaatcgaattaaacgttgttaaaggatttaaagtgcgaaaaatagtggcgaactgcagacgacaattgcacagtcagtaccccatttccattctcctgcgagtagaactctttcaatttgcatagacaaatgtggaaattctttgctgcaagttcagccgagacttcaagttgagtttcctgctcggctcggattctttaaatgaattaaataagattttctcttgtaacgtgcgttctcttttgagattaaaactaaatcaaaataataatttcatttgaacttattttcttacagccgcctaaagggaaaaagggcaaaaaaggcaaaaaattgccaggtaagtaaaaagctcgagtaaactgcactttagcatagatttatgaattcccaaatttaactactttgaacggtaattgaagtgaaggatatcagttaattgttgcaaaattcatttagtgccctctttaagaacaccggctgttaaagaccctcaacgagtagacaaactgaacaaggacgaggactgcctggcaattcattttcggaacattaatggcagcagggtgccaccactgggtagttgggtagttggatatctgggcagccggaatggcggacattggtcattcagggcaatttataaaggccaagcaaaacaattagtaaataacgataatctgcgtgaggagagaagcaaaagagaaggcaggacgcgggcgcaacgatagggacgataggagcgttgaggacaagaaattcccgatgcaattgctctgactgcagccttgcagtcagaaaatttaaaggcaaaaaatggggagggggggttccaaactgaccacttaatttgcacgagtgagcacactgtaggttccacaaatacccaaccatcctccgctctctgggatctctctaacgcacataattgtttctgtgcgtgcactgaaaaacgctcttactcgtggaacgcaaggaaactcctagtgtatcgtattattatagttatatcatcgatttgtttatgccacacacttatcttaacacatgtctctaaatagtttacacgcgctccttcactttgacttcaaattgtctttgcaatgcaatgtctttggtcttttttccaccgtgtggcatctagttcgtctcctggtagtcctgttggtcctgttggtcctagtagcccttcctaggccgagtcctggcaccagccggccaacgatgccaattacagaaaacaattacaacacggtctgctgttgggtcacatatgcgggcagcggctaagggaagggggggggggggcgtggcaggggtaaaaatggacgaaaaaggggcaaaagggggggggaggagagggtaaccaggcgtgccgctctcattaaatagccataaggacgggagcccccaatatgcggactaaacttttcacggacccctagcccaatattagaaaataaccaagacaaaaaaattcctgttcacacatgaacacgaatatatttaaagacttacaattttgggctccgttcatatcttatgtaaatgaatcgagagcgataaattatatttaggattttgttatctaaggcgacatgggtgcattgctcaaaaacatgtaatttaagtgcacactacatgagtcagtcacttgagatcgttccccgcctcctaaaatagtcccttagtgggagaccacagataaggtcctcgccgctcaagataggcagatgtgcccgagcgtgggacctcgataaggcggggactatttacgtaggcctctgcgtaggccatttactttaagatgcgattctcatgtcacctatttaaaccgaagatatttccaaataaaatcagtttcttacaaaaactcaacgagtaaagtcttcttatttgggattttacatttggtcaatcgagcctttaatcgactctgcagtttccccctaccaaaggtaaggaactcagagaaaggccagctcctttaagcatcttacagctaaaggtagcaaaaataagtgactcttgtttccccctaccaaaggtaaggaacagagtataaatataaaaagcaaaagatacaaaagaatcttttatgttttaaaacaagcaccttatagtctatagctaaaggttgctttgtgtaccattataaattgtggtaaggcgtgcttgaggccatacatcagcaattgtgaaattaaaaagtgcataacaaaagtgccttataaatgctctaatagcattaaatcagctcataaatagagtgcagtgtatatgccataagagcataaattaaataaaaagtgcctgaaaacagtgccttataaatgctctaatagcattaaatcagctcataaatagagtgcagtgtatatgccaaaagagcataaatgccgaaataaatggctaaaaaacaaaaaatctgactggactacaaaaataataaaacgtgccaaaaaaaaaaaaaaaaaaaaaaaaaatcatctttaaacatcgacggagccttaaagaagagaaggaagtcaaattcaaaggagcctctaccagcagcagaagcagcaacaacagcagcagcagaagcagcaacagcagtagcaacagcagcaacaacagcagcaacagcagcagcaacaacaacgacatcagctaagtcaaaacaagaattttctgtttatccaaacacacatatatatataaatacatataaaatacatatacacgtactatatatattaagaaattacaaaaaattttcaaaatgatgtcagaaaagactattcaattccttaagaagcagtccgaaattattttggaaattagaaagttggaagtaaaaccaacattaacagatgtagaaattctaaaattaaatgagcttcaaaaatgtttcattgctaatcatagcaatttgttaaagatcggcgttgtcgatcatgaatattttaacgcgaagcagtatgatttaataatgatggtgttagaaaaaattaaaaataaaaatgaaaaaattaagggcgagtcggtagaaaacactttccctaaatcaaacactgtccctaaatcaaaccctccccctacattaaaccttgaaatgcgtggtcaccctgaaaaagagggtatagcacaaaacaacgctttaaaagtagagcaggcatttcgtaataatgttggccaatttcgagtatatctagaagatacgtctaaactaatagacagtagtccagatttccttaaaataaggaaaaataaaattgaatttttatggcataaaatagataacctgattgaacaggtgaatagtcattttgagagttcgctattcgaagaagaaattagcgaacttgaatttgacaaacaaaatattcttacagccattaatagtcgactcagtggcacaataaataaagctgaaatgtcgacggttgttaaggcgaaggagttaccaaccctgcctaaaatacagattcccaccttctttggtgattccaaagaatgggatctttttaatgaactctttacagagctcatacatgtgagagaggatctcagtccttctctcaaatttaattatctaaagtcagcattaaaaggagaagccagaaatgtggttactcatttactgctcggctctggagaaaattatgaagccacttgggagtttttgaccaagcgatatgagaataaaagaaacatattctcagatcatatgaataggcttatggatatgccaaatttaaatttagaatccaataagcaaataaagacatttattgacacgattaacgagtcaatttatattataaaattaaaggcacaattaccagaagatgtggatgcaattttcgctcacataattcttcggaaattcaataaagaatcactcaatttatatgaaagccatgttaaaaagacaaaagaaatacaggcactttctgatgtcatggactttttagagcaaaggctcaattctatatcatcattctcacaggaagtaaaacctgtaaagaaaatgattaataataacaagaataaaaattatagtgacaattgtgcatattgcaaactaccagggcattatttaattcaatgccataaatttaaaataatgaatccagcagaacggtctgactgggtaagaaaaaatgggatttgcctaagatgtctgaggcatccgtttggtaaaaaatgtataagcgagcagctttgttcgacttgtcgtaaacctcaccacacgttacttcactttgcaggtcataatccagaaaaagtgaatacgtgtagaacaacaggtcaagccttgttggccacggccttgattcaagtaaagtcgaggtatggaggctttgaacaattaagagcattgattgatagtggctctcaaagcacaattatttcagaagagtctgcacagattctaaaattgaaaaaatttcggtctcatactgaaataagtggagtatcttccacaggaacgtgcatctccaagcacaaagcggttatttcgataagaaattctccgaaaaatttagaaattgaagcaattattctcccaaaacttatgaaggcacttccagtcaacacgattaatgttgatcagaaaaaatggaagaactttaaattagccgaccccgattttaataaaccgggtcgcattgatttaatcattggagcagacgtatatactcacattctgcaaaatggagttataaaaatagacggtctccttgggcaaaaaactgatttcgggtggatagtttctggatgtaaaaaatccaaaggaaaagaaaccattgtagccacaacaatagaaataaaagagttagatcgctactgggaagtggaagaagaagaaaaagatgatatcgagtctgaaatctgtgaaaataaatttatcaaaacgacaaaaaaagattcagatgggcgatacattgtgtcaattccattcaaggaggatgtcaccttaggagattcaaagaaacaagcgatagctcgttacatgaatctggagaaaaaactaaaaagaaatgaaaaacttaaggttgactacactaaattcatgaatgaatacatggatttaggacacatgattgaagtgagtgatgaaggcaaatattttttaccgcaccaggcagtgattagagattcaagccttacgaccaaattgagagtagtttttgatgcttcagcaaaaactacgaataacaaaagtttgaacgacataatgtgggttgggccacgagttcaaaaagatatttttgacattattattaaatggagaaaatgggaatttgttgtttcggcagacattgaaaagatgtaccgacaaattaaaatagataataatgatcaaaaatatcaatatattttatggagaaattctccaaaagaaaaaattaaaacatataaattaaccacagtcacttacggaactgcatctgcaccatatttggctaccagggttctggtagatattgcagataaatgtaaaaaccaagttattagtgcaataattaggaatgatttctatatggatgacctaatgactggagctgattcggtagaagaagctaataaattaataacattaattccccatgaattgcagaaagttggattcaacttaaggaaatggatttccaacaattccaaaatattaaccactgtggaggacacaggggacaataaggttctcaatattatcgaaaatgaatgtgttaaaactttaggactaaaatgggaacctcaaaaggatttatttaagttcagcgtaaattgtaatgatgaatcaaaaaatataaataagcgcgttgtgttatcaacgctagcaaaaatatttgatccgttaggatggttggcaccagtcacggtttcaggaaaactttttattcaaaaactttggataaataaaagtgaatgggatcaggaattatccatagaagataaaaattattgggaaaaatataaagaaaatttattattgttagagaatattcgaatcccaaggtggattaattcaaacagttcttcagtcattcagattcacggatttgcggacgcctccgaaaaagcatatgctgcagtagtctatgctaaagtaggacctcatgttaatataatagctagcaaaagtagagtcaaccctataaaaaataggaagacaattcccaaactcgagctgtgtgcagctcacctgcttagtgaattaatccaaagactaaaaggatcaattgacaatataatggagatctatgcttggagtgattccacgattaccttagcatggattaacagtggtcaaagtaagatcaaatttataaaaagaagaacggatgacattcggaaattaaaaaatactgaatggaatcatgttaagtcagaggataatccagcagatttagcatccaggggagtggattctaaccagttgatcaactgtgatttttggtggaaaggtccgaaatggctagcagacccaaaagaactttggcctcggcagcagtctgtagaagaacctgtcttaataaatacggtattaaatgacaaaatagatgatcctatttacgaattaatagaaaggtattccagtatagaaaaacttatacgtataatagcatacataaatagattcgtgcagatgaaaacaagaaataaagcctattcatcaattatttcagtaaaggagataagaatagcggaaacagttgttattaagaaacaacaagaataccagtttaggcaagagataaagtgccttaaaatcaaaaaggaaatcaagacaaataataaaatattgtcattgaatccatttttggacaaggatggggttctaagagttggaggaagattgcaaaattccaatgcagaatttaatgttaaacatccaatcattttagaaaaatgccacctaacaagcttattaataaaaaatgctcataaggaaacattgcatggagggataaacctaatgcgaaactatatccaaagaaagtattggattttcgggttgaaaaattcgttgaaaaagtatttaagagaatgtgtaacgtgtgcaaggtataaacaaaatacagctcagcaaataatgggtaacttgccaaaatatagagtgacgatgacattcccgtttcttaatactggaatagattacgcaggtccttattatgttaaatgttcaaaaaatcgtggccaaaaaacatttaaaggatacgttgctgtattcgtttgcatggccaccaaagccatacacttagaaatggtaagcgatctaacttcagacgcatttttagcagcactcagaagatttattgctagacggggaaaatgttccaatatctattcagacaacggaacaaattttgtaggagctgcaagaaaattagatcaagagttatttaatgcaatacaagaaaatataacgattgcagcgcagcttgaaaaggacaggattgattggcattttattcccccggcaggacctcacttcggaggtatttgggaagctggagttaagtcaatgaaataccatttaaagcgtataatcggcgacactattttgacttatgaagaaatgtcaactcttttatgtcaaatagaagcatgcttaaattcaaggccattatacactatagttagtgagaaggaccaacaagaagttttaacaccaggtcattttttaattggaagaccacctttagaaatagtcgaaccaatggaagatgaaaaaatcggaaatttggataggtggagacttatccaaaaaatgaagaaagatttctgggttaagtggaaaagtgaatatttgcatacgctccagcaaaggaataaatggaaaaaggaaattcctaatatagaagaagggcaaatagttttattaaaggatgagaattgtcatcctgcaagatggcctttaggaaaggtggaaaaggtgcataaggggaatgatgataaggtccgagtggctaaagtaaagatgcaggaaggatatatcactagacccattactaaaatttgtcccttggaaggaataaagtctgttgacaaaaatgaggctgaccaagagccaaaaagacgaactagagcgacatcgggaatgtccaagatcggaatcattatggcaatgttgttgtttgtgttaagttgtcaagtttctagcgcattacctaaagatatagcaccaagatattctatagacaaaataaataaaacctcagcaatatatctagacccgctaggagatgttgagattgtgagtacttcttggaatttggttatctattataaaatggatccatattttaaaatgttaacaaagggtaatgcgcttatacaaagtatgaggaaagtttgcgaaagacttcatagctttgaagagcaatgtagtctagtcttagataatatgcaaagtcagttatcggaacttgaagaaaacaataaattgtttatgatgcagtctagatctagaagcaagcgtgcccctttcgaatttatgggttccttgtatcatattttatttggtataatggatgaagatgatagagagcaattagaagaaaatatgaagaatttgttagataaccagaacaaccttgataaactaattcaaaaacaaacatctgtggttgattcaacttctaatctattaaagagaacaacagaagatgttaactccaattttagaagtatgcaaataagaattgagaacatgacagaagttcttaaagaaaattattatgtttataaggaatcaataaaattctttatgattacgaaacagctacactcattgattgaagaaggcgaaaaaattcaagcaggcattataagcctgttgattgatattaatcacggtaggctaaatacaaatattctcaggccaaatcagcttaaaaaagaaattgccaaaattcagcagagtctttcagagaacctagtaattccaggaaaacggtcaggtacggaacttaaggaggtgtatacactgttaacagccaggggtttattcatcgacgataaattgatcattagtgcaaaagtgcctctgtttagcaggcatccatccaaattgttcaggcttattccggtgccaattcgaaatgaagatcggataataatggtgcatacaacgtccgaatatttaatttataattttgagatagattcctatcacataatgacggaagccacattaaatcaatgtcagaaatggcaactaaataagagaatatgcaaaggaagttggccctggaattcagcgaatgataatgcatgtgagattcagcctctaaagccagataaagcggcgaactgcatctataaaacagtagtcgactctaaaagttactgggtagagttagaaaagaaaagtagttggttgtttaaggttcctgcgaattcaaaagtccgtctgcaatgtactggctctcaaattgaattgtttgatttgcctcagcaaggagttttaagcattgcgccatattgtacggcaagaaccgacgataaaattctagttgcccaccataacattcagtccgaaagtgaagaattattatcaacaccttatataggagaagttagtggagtgccgaagattatttgggatccgctgaaactatcaatattaaatcatactgaggaatttgaacgattgaataatgaaattaaatttatgaaagagaaccatcaaaaattgaaagatttacatttccatcatatttccggacatgctggattaattattgctttaatactaatgatagtattaataatatatttcatacggaaatgtgctgtgcaacaaagaatgcaagcaataacctttgcaggtccgttgccagtactataaatatcaatagtaaataaacaataaaataatataacaaataaaaatatacagtccactaatagaaaatgtacttctacatagaaaaagcaaaatgtttaaaataagttaattgagtacaaattgttgaattaaaaataatataaaccataattgtaatccaataaaattaaaagccagaaaaactaggcccattgaaatcttagttgcaaaataaatgaacatatatcaaataaatacagtccactactgttataaatgcaactaatatactaatgtacatctcagctttgctggccctttggcagaatgttcacacatgaacacgaatatatttaaagacttacaattttgggctccgttcatatcttatgtaaatgaatcgagagcgataaattatatttaggattttgttatctaaggcgacatgggtgcattgctcaaaaacatgtaatttaagtgcacactacatgagtcagtcacttgagatcgttccccgcctcctaaaatagtcccttagtgggagaccacagataaggtcctcgccgctcaagataggcagatgtgcccgagcgtgggacctcgataaggcggggactatttacgtaggcctctgcgtaggccatttactttaagatgcgattctcatgtcacctatttaaaccgaagatatttccaaataaaatcagtttcttacaaaaactcaacgagtaaagtcttcttatttgggattttacaattccctttggtaattagctttaaaagggctagtcgtatcctttgttttcatttcaaatcatttagctaaattttcgtctgcctatattcctgcttagtgctcatcgatggcgtggacacctcggcgatgactcgcgaccagctggaggcatttgctctccggctaaaagcggaaatggatcgtgagcgggaggagcgtaactacttccagttggagcgggacaagattcgcactttctgggagatcacgcgccagcagctgggtgagtgaataccaggggattttgccacctttcctccattttttttttttttttgttttgtttttagatgagacccgctacgagctgcagcagaaggacaaggagatcgaggccacgcaggatctggcggatatcgataccaagcatgtgatgcagcagatgaagcatctgcagtttgagaaccacaataggctcggtgaggttcgggctgaggcgatgacccaactaaagctggcgcaggagcaccatgttctgcaggaaaacgagcttcagcgggacaagcgacagttgcgccgaatgctgcgcgaaagaatggagatgagcgagatgcagctgcgccaaatggaggctcacttcaatgagaaactgctgtaggtattattatcgcagaaatatgaatcatattctcacgttttttttttcccattgttttcgtgcgctggcagagagcagcgcatcaccttcgaacgcgagcgcaaggacaacgagatgctgcacgaggagaaaatgatcgagcagaaggccaagctagaccttttctacggcacacaaatgttcgaggtagaggagcgaaagaaccagcagataaaggacctacaggaccaccatgacctagcctttaacgatatgaagaactattacaacgatatcacgcttaacaacctggcgctaattggcagcatgaaggagcagctagagcatctgcgcaagcaggccgagagatccgatagaatcgccgcagacacggcagctgagaatcggcgactgaaggagcctttggagcatgccaatatccagttgaacgagtatcgtcgcaaactggagttctacgagcgggataagcagcaattgagtcgcctcaagacgcgcaacactcggctggaaaagaaggtgaagggtctcacttgggaggcggaaactctgatcctgcgcaacgactcgctggtggcagaacgggagggcctgaaggagcgtttcaacgacgtgatcgtcgagctgcagcagaagacaggactaaagaatgtccttctggagcgcaagattgccgcattgatgcgcgaggatgagaagcgcagcattgtcctacacgaaacgattgccacctgcgctcccaatttcgccgaaaagttaaccagcttggatgaacgggtgggcaacatcatcgatgagaagaacaagataatccttgacctgcgctatgaggtaactaaggcgcgaaaggcacacgacgatctactggaaacctacgagtgcaagctcaagcaatatggtgtgcccactgacgagttgggcttcaagcccatcaggaatcgggaccaacagcagctgtacgtgtgcggtcctgcgggaataatcaccgagaataagtaggacctgctgaagaaaagtcatattcacaagtcggattaagaaaatatatcgaggcggggaaggaattcaaaaccagaagaaacaaaacttactccagaacaaaaagaagtaaaggggattagctgaaaactctagaagcaaaaggacaaacaaaaaaacgatcgaattatatttttatcaattagaaaacagttcatggaataatgggaaaggcataatggaacgcactttacttggtgatcatataaaaatatactctacaacgcaaatggagaatttcatcaagaagaaaacgaggaggaaaatccaatggaaaatcaaatgaaaaatccaatggaaaatccaatgaaaaatcccaccttggtaggcgcagaaaatgtaaattgttagtgctgcttggtttctttaagaatttattcattctgtgcctatctggagtgtattatgtaaactcccccgcccctcaaaaataatacggaagcaataaaacgtttaccaacgt

SEQ ID NO: 20 - DNA, 1365 bp, Drosophila melanogaster
  CDS (1)..(1362)
atgactcgcgaccagctggaggcatttgctctccggctaaaagcggaaatggatcgtgagcgggaggagcgtaactacttccagttggagcgggacaagattcgcactttctgggagatcacgcgccagcagctggatgagacccgctacgagctgcagcagaaggacaaggagatcgaggccacgcaggatctggcggatatcgataccaagcatgtgatgcagcagatgaagcatctgcagtttgagaaccacaataggctcggtgaggttcgggctgaggcgatgacccaactaaagctggcgcaggagcaccatgttctgcaggaaaacgagcttcagcgggacaagcgacagttgcgccgaatgctgcgcgaaagaatggagatgagcgagatgcagctgcgccaaatggaggctcacttcaatgagaaactgctagagcagcgcatcaccttcgaacgcgagcgcaaggacaacgagatgctgcacgaggagaaaatgatcgagcagaaggccaagctagaccttttctacggcacacaaatgttcgaggtagaggagcgaaagaaccagcagataaaggacctacaggaccaccatgacctagcctttaacgatatgaagaactattacaacgatatcacgcttaacaacctggcgctaattggcagcatgaaggagcagctagagcatctgcgcaagcaggccgagagatccgatagaatcgccgcagacacggcagctgagaatcggcgactgaaggagcctttggagcatgccaatatccagttgaacgagtatcgtcgcaaactggagttctacgagcgggataagcagcaattgagtcgcctcaagacgcgcaacactcggctggaaaagaaggtgaagggtctcacttgggaggcggaaactctgatcctgcgcaacgactcgctggtggcagaacgggagggcctgaaggagcgtttcaacgacgtgatcgtcgagctgcagcagaagacaggactaaagaatgtccttctggagcgcaagattgccgcattgatgcgcgaggatgagaagcgcagcattgtcctacacgaaacgattgccacctgcgctcccaatttcgccgaaaagttaaccagcttggatgaacgggtgggcaacatcatcgatgagaagaacaagataatccttgacctgcgctatgaggtaactaaggcgcgaaaggcacacgacgatctactggaaacctacgagtgcaagctcaagcaatatggtgtgcccactgacgagttgggcttcaagcccatcaggaatcgggaccaacagcagctgtacgtgtgcggtcctgcgggaataatcaccgagaataagtag

SEQ ID NO: 21 - protein, 454 aa, Drosophila melanogaster
MTRDQLEAFALRLKAEMDREREERNYFQLERDKIRTFWEITRQQLDETRYELQQKDKEIEATQDLADIDTKHVMQQMKHLQFENHNRLGEVRAEAMTQLKLAQEHHVLQENELQRDKRQLRRMLRERMEMSEMQLRQMEAHFNEKLLEQRITFERERKDNEMLHEEKMIEQKAKLDLFYGTQMFEVEERKNQQIKDLQDHHDLAFNDMKNYYNDITLNNLALIGSMKEQLEHLRKQAERSDRIAADTAAENRRLKEPLEHANIQLNEYRRKLEFYERDKQQLSRLKTRNTRLEKKVKGLTWEAETLILRNDSLVAEREGLKERFNDVIVELQQKTGLKNVLLERKIAALMREDEKRSIVLHETIATCAPNFAEKLTSLDERVGNIIDEKNKIILDLRYEVTKARKAHDDLLETYECKLKQYGVPTDELGFKPIRNRDQQQLYVCGPAGIITENK

SEQ ID NO: 22 - DNA, 10387 bp, Drosophila melanogaster
cgattgaaaatgtaagtgcatcctgtcagctgctctccaagaaaataccaaacgcgctgagtggaaaaacgagctagggtttgaatagttggaatatagcaaaggtaaggcaataatctgaataatatatccaccatttttgcaagaccatgtagatagtgtactcatgacccaaagtaaccccttaaatttccctttaattaagggatttatttacccgatggcccgtcaatgtttgctctgatatttgcccaccatcaccacttagactaatcaaaaagcctctagttttgtcaactcatccaattgggtgtagaaataatcagagctttttttctcacctaaggcttagtacctctaatttcattagacggaaatcgtccaaatcatctgataagtatctcttcaatgtaattaaatgttcaatacccgcctagaaatatctgcgcactatgagaagccttgcagtttgaatcctttgctcgtcgaaagcagttcgaaaatggggctggcggaagttaagtaagtctggatttacaaaggcttggcattatgcaaatttctccagtcgagtggcgaaaataaacaaaccattaattagacagttccagttaatgggcccgcgaaagggagtcccggcttcgattccctcgggcggcagtggctataaatgtgtagaatgacaaatggccattgacttgctagccagctgtacttttatacatacagtatatatatacggctcagtgcgtgtaaattggccaagggttactgctaaattagccctagttgcagttacgcttcagttctagtccgacacagttgtatctagtgtgaatcgtcgtatcgctcgtatctcctatatctggcgatatcatgtgacacacggtttatctaaattgctttgctgtcggtttacgcgtttgcaaaccgtttaacccaatcaatatctaattagtccactgattgctgcccagcccgatgatgtttgataagttgcaaaatacaaacgaggcactaaccagcgcgtacagtcgcgaccagcggccctttatgattacagctccggccacggccacggaacgcaaccagagttccaagagcaccaggatgcccaagctctacaacggagtctacagcggtcagtgcggcgccctatcgccacctgacctcatggaggcccagccgaagctacttcccaagccaaggagcaacagcagcggcagcaccggccggaacagcaaggtatgaaacagcaaaaaagaaaaatgattcttttttgtataatagggatagcttttatttaaaatctgtgccaattcctcacctatggtgacgccaggtagatgtgctcttctcagataaaaaaacccccgatgagattcgaacttacgatctcctgtttactagacaggcgctttgcccaactaagccacggcgccacattttgaaatttatgataatattattttattaaaatcatgttaatattggttttaaagcagtttgaacctagaaaaatatattaattatattctaaattaagaagtgaagtggattaaatatactagacctattcgaatttcagttcgaactcgttcgataatattccattttataattagccagcacttataaataatttatgacttaacataacttaaccaatttgacatctttttgcagtattggatattttcaatgataatcgagcgcagtgcgggtcccaagcggattgaaatcgatggcgatgatgcggacacgccgctggaggccatcctgccagccgaaccgccggcggaggtctgcctcttgcgtgacagccccttcaggatattgcgggtgagtagatccagaatgggttccatgcaggtgcgggccggcaattctccaaagtgaagacccgaggcaattcccgcagcgagagccatgataaatgcttctcgatgtgcacctagcctcgtgttttgaatgttcgagaagcggccaaggaagcccgatagatattattcaataaaatgtcaatcgttttttttataaaaaatataaaagaacacttatgaagacttaagcaaatataagttattttaatgacttatattcaagcagttaacagatccattgaggatatttttacctagaagtgattggagcaacaagtcctttttcgcattcagcgccagagaaagaaagattgaaattgaaaccaaaattgaccggcgagttcaacaaaataacacgaagcgcatccttctccaaaaggacctgtgccacacgcacccgaagttgccgccagttggctataaaagcaagactgtttggtccgagatttcagtcattcagcgactcttcccgcgtggactcaacctttttttttcctctggatatatttactctccgtcgagcttaccaaaaatcaagttaacttggcagcaaatcagtggcacagcaatttcatttattacatttgtggcagttaactaactcaataacgacttgtgctaaagaaaaaaaagtttatacacaaaaccgtgcaacaccaaggatattttacacatttaaaattcgtatgacaagtgaacgcgaattttgaaaaattaactttaatacattccacagtacaaatataacattaaatatacaaaatattgctcaaatagtgataacaactgaacttgtttttttttttataacaataacaatatatacaaaactgcaatttatgaaacgtacgcatatcaaagctgcaagtcttctgattatcgcaatctaattgagtttcgatcggaattaaaacaccaaacgaactgcaaaacagagctgaattcataagatgagctaaaggactcagttcataagttcgcataaccaaactacaagctgtcgtaacgaaacgctatttctggaatgcaaaacgcattctaattgaatgtccactggcattaaggacccattgagtcatcacgtaaaggcagaaaatcagaaactgaattcagaaggcacaaggcgaacgcatatggaaatcatattggaaaaaatccataacaagtcaaggctctctcgaaacttcgtccgatgttgccagctgagctgtaaggacaataagagttaatccactaactaaacacaaatgtaattaaagtcgacaatgacttcgggcgacaaggagactcctaagagggaagatttcgccagtgcgctgcgattcttgatgggcggctgtgcccgcgaaccggaaatgactgcaatggcgccgcttaacctgcccaaaaagtgggcacgcatcctgcggatgtcctcgactccaaagataccaatcgtggactatctggaggtaaaactgcagccggcgaactcattaaattagccacgcgcgaatcacaaaatgacctctctgggttggctaaaagtttcatttcaccccctccttttaagtgattcacagaattatactcctggacatttccagaaaaacgctgttcaatacagtgttttcaaattctttttttttttttttttgaagaaaacaggaaaggcaataagacaatcaaattactcagtcgccggaaggcgttaatgtcctaaaatctattgacattttgctgtaggacaatgttttgcactttctttgctttgcactacgaaaggcaaacagaaagttcttagagaaagtaaatgaaactaaaagaacttcgccttgaactagcaaagcacctttataacacaaaaattaaaagaaaaaataaattgcatgcaaaagaattttgtattgttatgacattttaatttattaacccgaaggaagttaaagactcggaatactaggaatgttcctcacctccgaacaagagttcaaaatcagttttataaattaaaatttttcttaattaagtatttttcccagtgctggcaaaccaaacatagcttaatgttttaattgatgttgctgcaaagtgaaagacacctgtttttcctgtttcaggcggctgagtccggaaaccttgacgacttcaagcgactcttcatggcggacaactcgcgcattgctttaaaggatgcgaaaggacgaacggctgcccatcaggcggcggcccgtaatagggttaacattttgcggtacattcgcgaccagaatggcggtaagtgaacggataaagccattaacccggactccttatcaagcgtgatttacagactttaatgcgaaggataatgccggcaataccccgctccacatcgccgtggagagcgatgcctacgacgctctggactatctattgtccatgtaagtggctcaagccatggttgatttagcccgattgaacttcaaaatatatcttgccgacaatccttacagcccagtggatacgggagtgctgaacgagaagaagcaggcaccagtgcacttggccaccgagctgaacaaagtgaagtcccttcgggtgatgggtcagtaccgcaatgtcatcgatattcagcagggcggcgaacatggacgtaccgctctgcacttggccgccatctatgatcacgaggagtgcgctcgcatcctggtaagtggtgtgagttttggccaatcatttcatttgattattttttatgttcggccttgcttttgtatttactcatgcattttatttgcattggtttgatttatattaagccattaatcaacttgattcacccaccatcgtcctttcgaaaaacacactcatccgtacaaaagtttaatttatcatagagaacatgaaaaataaaggttggtatatgcagtagaagaaaattgaaattgttttgcctgcttttatactagacccaaatattaaagagtgtgtctgtgtgcgtgtgtgtatgttcctagtgctagcatattatattagacgcatttatatttgtttacaataactcatcgtatcttccagataactgagttcgatgcatgcccacgtaagccctgtaacaatggttattatcccatacacgaagcggccaagaatgccagctccaagacaatggaggtcttcttccaggcaagttatcctttccatttcccgattcgcacttgtcccagctgcatgctacaactacgagtatgtcatctcatccagctttgatcctattgtggaagctatccccattggacactgacccgatttgcttgattcctacaactcctacccctcttacttactttcttacttacatcccatccagacctttcgtgcactctcttcggttttgggtttatttttatttggtttttgattttgctttgctccttgattgaagttgcgtatacgccgcgttgtcagcgacctctttaaaaagtatcaaatttgttatgggtcacactgtcgctgtcgagagttgaaaggcatggagaataacatgttttccacaaaattttaagctgcagaaaaatatcagatgttggataactcaggcggggtcggtcattcactttggacaacagtttactggtcgccttaagacacttctaggatgcccaaaagttaaagcgttgcaaaaaaaaagaaaaaaaaaacagagtgatgtgttattttgattgcttttcttgacttggtttacttaaatgtgtaatcaattatagaaggaatgttcttagatataagtatgagcttagactgcactgccgcttcatagaattacttgttcaagttttttaatagtaatttaatcttttgtgtagctattgttattaatatattgacacctgcttttaaaagctgtcttaagaccattgaggataaatacgagaacggtgacgcaaacaagtctgttaatttctttttttcacaattaaatctctgcaggaatgagctcatctaatgtttttattcattttccagtggggcgagcagcgcggctgcacccgcgaggagatgatatccttctacgactcggagggcaatgtgccgctccattcggctgtccatggtggcgacatcaaggctgtggagctgtgcctcaagtccggggccaagatatctacgcagcaacacgatctctcgacgccagtgcacctggcttgtgcccaggtgagtgtatgctttggatgcacaggtgtgctgatgactgggaaatccttcagcggcgaccaactgacagtctgccaagcaattgaaaactctcatatttccacgtggagttgagcaaacaaagcgttcttggccagtaagcgaaaggaaaacagcattaaagcatatccaatgggcggtgaaaagtggctgtggtggtttcctttcggtctccttcggattgcctcgtgtcttggtgtccttagttgactttaatttgacaaattcttatgcaaattcactaagaagaacgtgactttgccgttaacaaaacaaagcacttttcatgttggggaaatacatactacggatgaatgattccgacatggttttccatttccatttccgcttcttgttctttttgtttttttttttgtctagggagccatagacattgtaaagctcatgttcgagatgcagccaatggagaagcgactatgtctgagttgcacggatgtgcagaagatgacgccgctgcactgcgcctccatgttcgatcatccggacattgtgtcctatctggtggccgagggagcggacatcaatgccctggacaaggagcatcgctctccgttactgctggcggcatctcgtagcggttggaaaacaggtgaccttagttaacccttttatatatctattgtgtaaacttggctttccatttttccagtccacctcctgattcgcctgggggcgtgcattagtgtcaaggacgccgccgcccgcaatgtgctgcacttcgtcatcatgaacggcggccggctgacggacttcgcggagcaggtggccaattgccagacgcaggcgcagctgaagctgctgctcaacgagaaggacagcatgggctgctcgccgctgcactacgccagtcgggatggacacatccgttcgttggagaacctcattcgactgggagcctgcatcaacctgaagaacaacaacaacgagagtccgctgcactttgccgcacgctacggaagatacaatacggtgcggcagctcttggattccgagaagggatccttcatcatcaacgaaagtgacggtgcagggatgacaccactgcacatatcctcgcagcaaggacacacgcgagtggtgcagctgctactcaatcgaggagccctgctccatcgggaccacaccggacgcaatcctctccagctagcggccatgtccggatacaccgagaccatcgagctgctgcactcggtgcactcgcatctgctcgatcaggtggacaaggatggggtgagtgataatacttggcaacttggttaatcaaggtaaccattaaacatggctaatccccattagaacaccgctcttcacctggccaccatggagaataagccccatgcgatctccgtgctgatgtctatgggctgtaagctggtctataacgttctggacatgagtgccattgactatgccatctactacaaatatccggaggctgccctggccatggtcacccacgaggagcgggccaacgaggtgatggctctgcgttccgacaagcatccgtgcgtgaccctcgccttaattgcctccatgcccaaggtattcgaggcggtgcaggacaagtgcattaccaaggccaattgcaagaaggactcgaagagtttctacgtaagtttgtcgttttcccagggtgaagcccaatcatcatctcattccatcgaagctaattgatttctattcgcccccattgacatcgccgtgaccagatcaaatactctttcgcattcttgcaatgcccctttatgtttgccaagattgatgagaaaaccggagagtcgattacgaccgccagtcccattccgttgccggctttgaatgtaagcggaaactggaatccttcccagatctgttgcatgcgtgtttttttaatgttgctcctccttcctggccttgatacgtgatcttggggttattgtgggctgggatgtagcccacaatcgagtatagaatgaggtttagcgggcacatcatgatccataattcatattaatggctaagttccatacatcgatactcattacttggttaccatccagataaaatattcgttttggccctaccaaaagacacccgaacagattgaggccaagcgcaaagagttcaatgaccccaagtggcgacccgcgcctttggccgtggtgaacgtgagtgtccccgaactctcttaaaggtcccagagctctgcaattgtgaaccgtcccccaaactaacccactaaccgactcactttccggcttttgtatcttgtatgttgtgtgcgtgtttattagactttatgctacccatttctgatatatgtttccctataatccctcgatcagaccatggtaacacatggcagggtggagctgctggcccatccgctcagtcagaagtatctgcagatgaagtggaactcctacggcaagtactttcacctggccaacctgctaatctactcgatattcctggtctttgtaaccatctactcttcgctgatgatgaacaacatcgaactgaaggctggggacaacaagacgatgagtcaatactgcaatatggggtgagttgagatcatagttacccgaaaacatatagcaacacctccatttttcagatgggagcagctgaccatgaatctctcgcagaacccgtcggtggcatcacagattcgtttggattcctgcgaggagcgtataaatagaaccactgcaatacttttctgtgcggtggtcatcgtggtctatatactgctcaactcgatgcgggaactaatacagatataccagcagaaattgcactatatcctggagacagttaatttgatatcctgggtgctgtacatctcggctttggtgatggtaacaccggcatttcagccggatggaggaatcaataccattcattactcggccgcttcaatagcagtctttctgtcgtggttccgattgctactgttcctgcaaagattcgaccaggtcggcatctatgtggtcatgttcttggagattctgcagacgctcattaaagtgctgatggtattctccatacttataatcgcctttggtctggctttctatatactactttcaaaggtaggtgcttttatttggatttttcgagtgagatatttcctacttatcccttagattattgacccccaaccgaaccacttgtccttctccaacatacccatgtccttgctgcgaactttctcaatgatgctgggcgagctggactttgtgggtacctatgtgaacacctactatcgggatcagttgaaagtgcccatgacatcctttttgattttgagtaagtattgtttacaccattacatttccttcaattgatattaaatttattatgtcaggtgtctttatgatccttatgcccattcttctgatgaacttgctcatcggtttggccgtcggcgatattgagtcagtgcgtcgcaatgcccagctcaagagactggccatgcaggtggtgctccacacggagctggagaggaagttgccccatgtctggctgcagcgagttgacaagatggagctgattgagtatcccaatgaaaccaagtgcaagctgggcttctgcgatttcatcctgcgcaagtggttctcgaatccattcaccgaggattgtaagttgtctgggaaaagatctggtactaggtccatccaatacactttcaacttctatgcagcctccatggacgtcatctccttcgacaacaatgatgactacatcaacgcagaattggaacggcagaggcgaaagttgcgcgacataagtcgcatgctggagcaacagcaccatctggttcggctcattgtccaaaagatggagatcaagacggaggcggatgacgtggacgagggtatatccccaaacgagttgcgatccgtcgtcggtttgagatcggcaggcggaaatcgatggaactcgccgcgagtccggaataaactccgagccgccctgagcttcaataagagcatgtagatcccttcgttcgaaggaacttggctttataacaaaatctgtgtatcgtccttagtagaaaaattgcaccatttttaatatatctctatgtgtgtgtaaatcgttgtcccacagtgacgtggatcgtgtgtgtatgtgacgatcgtattgtgtgaggtcctgaaaggaataatacatcgaatttataggcaaggatcatgggctgttctcgtaacttgattaagctgtggaattcaacagatctattgtagttttaaatgcaatttgcaaaagtgtgtattgtt

SEQ ID NO: 23 - DNA, 3588 bp, Drosophila melanogaster
  CDS (1)..(3585)
atgcccaagctctacaacggagtctacagcggtcagtgcggcgccctatcgccacctgacctcatggaggcccagccgaagctacttcccaagccaaggagcaacagcagcggcagcaccggccggaacagcaagtattggatattttcaatgataatcgagcgcagtgcgggtcccaagcggattgaaatcgatggcgatgatgcggacacgccgctggaggccatcctgccagccgaaccgccggcggaggtctgcctcttgcgtgacagccccttcaggatattgcgggcggctgagtccggaaaccttgacgacttcaagcgactcttcatggcggacaactcgcgcattgctttaaaggatgcgaaaggacgaacggctgcccatcaggcggcggcccgtaatagggttaacattttgcggtacattcgcgaccagaatggcgactttaatgcgaaggataatgccggcaataccccgctccacatcgccgtggagagcgatgcctacgacgctctggactatctattgtccatcccagtggatacgggagtgctgaacgagaagaagcaggcaccagtgcacttggccaccgagctgaacaaagtgaagtcccttcgggtgatgggtcagtaccgcaatgtcatcgatattcagcagggcggcgaacatggacgtaccgctctgcacttggccgccatctatgatcacgaggagtgcgctcgcatcctgataactgagttcgatgcatgcccacgtaagccctgtaacaatggttattatcccatacacgaagcggccaagaatgccagctccaagacaatggaggtcttcttccagtggggcgagcagcgcggctgcacccgcgaggagatgatatccttctacgactcggagggcaatgtgccgctccattcggctgtccatggtggcgacatcaaggctgtggagctgtgcctcaagtccggggccaagatatctacgcagcaacacgatctctcgacgccagtgcacctggcttgtgcccagggagccatagacattgtaaagctcatgttcgagatgcagccaatggagaagcgactatgtctgagttgcacggatgtgcagaagatgacgccgctgcactgcgcctccatgttcgatcatccggacattgtgtcctatctggtggccgagggagcggacatcaatgccctggacaaggagcatcgctctccgttactgctggcggcatctcgtagcggttggaaaacagtccacctcctgattcgcctgggggcgtgcattagtgtcaaggacgccgccgcccgcaatgtgctgcacttcgtcatcatgaacggcggccggctgacggacttcgcggagcaggtggccaattgccagacgcaggcgcagctgaagctgctgctcaacgagaaggacagcatgggctgctcgccgctgcactacgccagtcgggatggacacatccgttcgttggagaacctcattcgactgggagcctgcatcaacctgaagaacaacaacaacgagagtccgctgcactttgccgcacgctacggaagatacaatacggtgcggcagctcttggattccgagaagggatccttcatcatcaacgaaagtgacggtgcagggatgacaccactgcacatatcctcgcagcaaggacacacgcgagtggtgcagctgctactcaatcgaggagccctgctccatcgggaccacaccggacgcaatcctctccagctagcggccatgtccggatacaccgagaccatcgagctgctgcactcggtgcactcgcatctgctcgatcaggtggacaaggatgggaacaccgctcttcacctggccaccatggagaataagccccatgcgatctccgtgctgatgtctatgggctgtaagctggtctataacgttctggacatgagtgccattgactatgccatctactacaaatatccggaggctgccctggccatggtcacccacgaggagcgggccaacgaggtgatggctctgcgttccgacaagcatccgtgcgtgaccctcgccttaattgcctccatgcccaaggtattcgaggcggtgcaggacaagtgcattaccaaggccaattgcaagaaggactcgaagagtttctacaccatggtaacacatggcagggtggagctgctggcccatccgctcagtcagaagtatctgcagatgaagtggaactcctacggcaagtactttcacctggccaacctgctaatctactcgatattcctggtctttgtaaccatctactcttcgctgatgatgaacaacatcgaactgaaggctggggacaacaagacgatgagtcaatactgcaatatgggatgggagcagctgaccatgaatctctcgcagaacccgtcggtggcatcacagattcgtttggattcctgcgaggagcgtataaatagaaccactgcaatacttttctgtgcggtggtcatcgtggtctatatactgctcaactcgatgcgggaactaatacagatataccagcagaaattgcactatatcctggagacagttaatttgatatcctgggtgctgtacatctcggctttggtgatggtaacaccggcatttcagccggatggaggaatcaataccattcattactcggccgcttcaatagcagtctttctgtcgtggttccgattgctactgttcctgcaaagattcgaccaggtcggcatctatgtggtcatgttcttggagattctgcagacgctcattaaagtgctgatggtattctccatacttataatcgcctttggtctggctttctatatactactttcaaagattattgacccccaaccgaaccacttgtccttctccaacatacccatgtccttgctgcgaactttctcaatgatgctgggcgagctggactttgtgggtacctatgtgaacacctactatcgggatcagttgaaagtgcccatgacatcctttttgattttgagtgtctttatgatccttatgcccattcttctgatgaacttgctcatcggtttggccgtcggcgatattgagtcagtgcgtcgcaatgcccagctcaagagactggccatgcaggtggtgctccacacggagctggagaggaagttgccccatgtctggctgcagcgagttgacaagatggagctgattgagtatcccaatgaaaccaagtgcaagctgggcttctgcgatttcatcctgcgcaagtggttctcgaatccattcaccgaggattcctccatggacgtcatctccttcgacaacaatgatgactacatcaacgcagaattggaacggcagaggcgaaagttgcgcgacataagtcgcatgctggagcaacagcaccatctggttcggctcattgtccaaaagatggagatcaagacggaggcggatgacgtggacgagggtatatccccaaacgagttgcgatccgtcgtcggtttgagatcggcaggcggaaatcgatggaactcgccgcgagtccggaataaactccgagccgccctgagcttcaataagagcatgtag

SEQ ID NO: 24 - protein, 1195 aa, Drosophila melanogaster
MPKLYNGVYSGQCGALSPPDLMEAQPKLLPKPRSNSSGSTGRNSKYWIFSMIIERSAGPKRIEIDGDDADTPLEAILPAEPPAEVCLLRDSPFRILRAAESGNLDDFKRLFMADNSRIALKDAKGRTAAHQAAARNRVNILRYIRDQNGDFNAKDNAGNTPLHIAVESDAYDALDYLLSIPVDTGVLNEKKQAPVHLATELNKVKSLRVMGQYRNVIDIQQGGEHGRTALHLAAIYDHEECARILITEFDACPRKPCNNGYYPIHEAAKNASSKTMEVFFQWGEQRGCTREEMISFYDSEGNVPLHSAVHGGDIKAVELCLKSGAKISTQQHDLSTPVHLACAQGAIDIVKLMFEMQPMEKRLCLSCTDVQKMTPLHCASMFDHPDIVSYLVAEGADINALDKEHRSPLLLAASRSGWKTVHLLIRLGACISVKDAAARNVLHFVIMNGGRLTDFAEQVANCQTQAQLKLLLNEKDSMGCSPLHYASRDGHIRSLENLIRLGACINLKNNNNESPLHFAARYGRYNTVRQLLDSEKGSFIINESDGAGMTPLHISSQQGHTRVVQLLLNRGALLHRDHTGRNPLQLAAMSGYTETIELLHSVHSHLLDQVDKDGNTALHLATMENKPHAISVLMSMGCKLVYNVLDMSAIDYAIYYKYPEAALAMVTHEERANEVMALRSDKHPCVTLALIASMPKVFEAVQDKCITKANCKKDSKSFYTMVTHGRVELLAHPLSQKYLQMKWNSYGKYFHLANLLIYSIFLVFVTIYSSLMMNNIELKAGDNKTMSQYCNMGWEQLTMNLSQNPSVASQIRLDSCEERINRTTAILFCAVVIVVYILLNSMRELIQIYQQKLHYILETVNLISWVLYISALVMVTPAFQPDGGINTIHYSAASIAVFLSWFRLLLFLQRFDQVGIYVVMFLEILQTLIKVLMVFSILIIAFGLAFYILLSKIIDPQPNHLSFSNIPMSLLRTFSMMLGELDFVGTYVNTYYRDQLKVPMTSFLILSVFMILMPILLMNLLIGLAVGDIESVRRNAQLKRLAMQVVLHTELERKLPHVWLQRVDKMELIEYPNETKCKLGFCDFILRKWFSNPFTEDSSMDVISFDNNDDYINAELERQRRKLRDISRMLEQQHHLVRLIVQKMEIKTEADDVDEGISPNELRSVVGLRSAGGNRWNSPRVRNKLRAALSFNKSM

SEQ ID NO: 25 - DNA, 1542 bp, Drosophila melanogaster
ataggttcgaaatgtaaacaaacatggaaactgaaactgaagcgcagaaattgtattagctatggccaaaaatgtaaagaaattaacgttggccaacaagttggcaaaacgcggagatcttataaaacccacggaaataaaaagtcgtccaaatagggcggataacgtgagggtaggcggaaatgcatgataaacaatgaaattgatactcaagacgcagtttctaaatcttccaggatatagaggatctggttggtgtatctgctggcgcagcaggatcgagtagctcagtgttcttctcccccgtgcaaacccgcaagcagcgtctcttaaatggggaagccgtgaagaaaactaacattaaaatggagcccttgtctccagctcgagtggcacccaaaaagtttagaaaggatgatcgcatggtaaccagagttcaaatgggttcggctactgtggtcatatgcaaggtggtccctgatgctgtggactccccagtgagagtggataagataagagaggaggtggaaccccaaataaaacaggaagctccggaggattccccttctttttctgaggtgcaagcaccacatcctttgtggtttaaccacctagaaaacattcgcattatgagaaactccagaactgcgccagtggacaccatgggatgccaccgatgcgccgatttaaaagcagactcgaaggtatttccaataagtggaattaagtacatagaagaatatgttacaagcaaacaacttccttgcagacccagcgcttccaaaacttagtggcattgatgctgtccagtcaaaccaaagaccaaactacttatgaagccatgaatcgccttaaagaccgaggtctaactccactgaaagtaaaggaaatgccagtgactgaactcgagaatttactgcatcccgtatccttctacaaggtttggtggaattaataataaataaatatatcgcacttactctcgatctcttccagaacaaagctaaatacctcaagcaaaccgtggagatactgacggacaagtatggttcggacattccagataatgtcaaggaccttgtagctcttccaggagttggccccaagatggcccatatatgcatggcagtggcctggaacaagataaccggaatcggagtggacgtccacgtccaccgcctttctaacaggctgggctgggtgccgaagcccaccaaggagcccgagcaaacccgtgtggcgttggagaagtggctgccattcagcctctggtcagaggtcaatcatttgttcgtgggattcggacaaaccatttgcaccccagtgaagccaaattgtggggagtgcctgaacaaggacatttgtccttcagcccatgctgaaactaaagaaaaaaggaagaaagaccgataggaaagaggttcagcgaacgctgaaaaaggttttaggtttctaagtgtttctgaaattattagtgctaagaaaaatattgtgttacccataagaaaataaatgaattattaagggggctgtaagtcctgt

SEQ ID NO: 26 - DNA, 1167 bp, Drosophila melanogaster
  CDS (1)..(1164)
atggccaaaaatgtaaagaaattaacgttggccaacaagttggcaaaacgcggagatcttataaaacccacggaaataaaaagtcgtccaaatagggcggataacgtgagggatatagaggatctggttggtgtatctgctggcgcagcaggatcgagtagctcagtgttcttctcccccgtgcaaacccgcaagcagcgtctcttaaatggggaagccgtgaagaaaactaacattaaaatggagcccttgtctccagctcgagtggcacccaaaaagtttagaaaggatgatcgcatggtaaccagagttcaaatgggttcggctactgtggtcatatgcaaggtggtccctgatgctgtggactccccagtgagagtggataagataagagaggaggtggaaccccaaataaaacaggaagctccggaggattccccttctttttctgaggtgcaagcaccacatcctttgtggtttaaccacctagaaaacattcgcattatgagaaactccagaactgcgccagtggacaccatgggatgccaccgatgcgccgatttaaaagcagactcgaagacccagcgcttccaaaacttagtggcattgatgctgtccagtcaaaccaaagaccaaactacttatgaagccatgaatcgccttaaagaccgaggtctaactccactgaaagtaaaggaaatgccagtgactgaactcgagaatttactgcatcccgtatccttctacaagaacaaagctaaatacctcaagcaaaccgtggagatactgacggacaagtatggttcggacattccagataatgtcaaggaccttgtagctcttccaggagttggccccaagatggcccatatatgcatggcagtggcctggaacaagataaccggaatcggagtggacgtccacgtccaccgcctttctaacaggctgggctgggtgccgaagcccaccaaggagcccgagcaaacccgtgtggcgttggagaagtggctgccattcagcctctggtcagaggtcaatcatttgttcgtgggattcggacaaaccatttgcaccccagtgaagccaaattgtggggagtgcctgaacaaggacatttgtccttcagcccatgctgaaactaaagaaaaaaggaagaaagaccgatag

SEQ ID NO: 27 - protein, 388 aa, Drosophila melanogaster
MAKNVKKLTLANKLAKRGDLIKPTEIKSRPNRADNVRDIEDLVGVSAGAAGSSSSVFFSPVQTRKQRLLNGEAVKKTNIKMEPLSPARVAPKKFRKDDRMVTRVQMGSATVVICKVVPDAVDSPVRVDKIREEVEPQIKQEAPEDSPSFSEVQAPHPLWFNHLENIRIMRNSRTAPVDTMGCHRCADLKADSKTQRFQNLVALMLSSQTKDQTTYEAMNRLKDRGLTPLKVKEMPVTELENLLHPVSFYKNKAKYLKQTVEILTDKYGSDIPDNVKDLVALPGVGPKMAHICMAVAWNKITGIGVDVHVHRLSNRLGWVPKPTKEPEQTRVALEKWLPFSLWSEVNHLFVGFGQTICTPVKPNCGECLNKDICPSAHAETKEKRKKDR

SEQ ID NO: 28 - DNA, 1330 bp, Drosophila melanogaster
gcaaaacgcgaactagttccgcagcttgtgcaatgttttgtaatcgtatttgcaaatttattcaattaattaattataaataaaaaatgctgcgccgcgcacaatgcctgctccggctgcacggcaatggaggccattcgctggtcagccgctttcgaaactacgctacggacgagggaaatccgaaacagaacccgaatccaaatcccagggcacaaaaacccggcaccaaaaacctgccggctttaaggaacccctttgccgccgcccaggacaggacgaaaaacagctacctgaccatggtggagatattccaggagcgcgacgtccaccgtcggaaccatgtggagttcatctacgcggcactcaagaatatggcggatttcggggtggaaagagacttggaggtctacaaggccctgatcaacgtgatgcccaagggcaagttcatacccaccaacatgttccaggcagagttcatgcactaccccaaacagcagcagtgtatcattgatctgcttgagcagatggaagattgcggggtgatgcccgatcacgagatggaggcgatgctgcttaatgtgtttggcaggcagggacatccactgcgcaagtattggcgcatgatgtactggatgccaaagtttaagaacctatcaccgtggccactgcccgatcctgttccggatgatacattggaaatggccaagctggcgctagagcggatgtgcacggtcgacctgcggtccaaaatcacggttttcgagaccagcgagctgaaggatgccattgacgatacgtggatcgtgagcggaatgagtcccgagcaggagaaactgctgcgggagcactctcgccagaaagctctgtacatcgagggaccctttcacatatggcttaggaatcgccggatcaactactttaccctgcgcgctgatgcagattctgagttcctgtctgaattagatgagcggcagctggacgaggacgatgtctcccacatcgaagtacccttctttggtcgtgctccaccaaggcgacacaaccagctgggaaagctgcgctctgtccaccaacaggacgacggaaccattatggccatctgtgccacaggcacctccacaaaggactcattgctctcgtggattcgcctgttggaagcgaacggaaatccctccataggagaggtgcccgtcctcttccggttcacatccgaagtgccagccaaggcggaggaaattgaaggtggcgccagtgtcccggcaacaagtgataacagtagtcaagatgagcacattagcagtagacagaaataaacaaattgttgagc

SEQ ID NO: 29 - DNA, 1230 bp, Drosophila melanogaster
  CDS (1)..(1227)
atgctgcgccgcgcacaatgcctgctccggctgcacggcaatggaggccattcgctggtcagccgctttcgaaactacgctacggacgagggaaatccgaaacagaacccgaatccaaatcccagggcacaaaaacccggcaccaaaaacctgccggctttaaggaacccctttgccgccgcccaggacaggacgaaaaacagctacctgaccatggtggagatattccaggagcgcgacgtccaccgtcggaaccatgtggagttcatctacgcggcactcaagaatatggcggatttcggggtggaaagagacttggaggtctacaaggccctgatcaacgtgatgcccaagggcaagttcatacccaccaacatgttccaggcagagttcatgcactaccccaaacagcagcagtgtatcattgatctgcttgagcagatggaagattgcggggtgatgcccgatcacgagatggaggcgatgctgcttaatgtgtttggcaggcagggacatccactgcgcaagtattggcgcatgatgtactggatgccaaagtttaagaacctatcaccgtggccactgcccgatcctgttccggatgatacattggaaatggccaagctggcgctagagcggatgtgcacggtcgacctgcggtccaaaatcacggttttcgagaccagcgagctgaaggatgccattgacgatacgtggatcgtgagcggaatgagtcccgagcaggagaaactgctgcgggagcactctcgccagaaagctctgtacatcgagggaccctttcacatatggcttaggaatcgccggatcaactactttaccctgcgcgctgatgcagattctgagttcctgtctgaattagatgagcggcagctggacgaggacgatgtctcccacatcgaagtacccttctttggtcgtgctccaccaaggcgacacaaccagctgggaaagctgcgctctgtccaccaacaggacgacggaaccattatggccatctgtgccacaggcacctccacaaaggactcattgctctcgtggattcgcctgttggaagcgaacggaaatccctccataggagaggtgcccgtcctcttccggttcacatccgaagtgccagccaaggcggaggaaattgaaggtggcgccagtgtcccggcaacaagtgataacagtagtcaagatgagcacattagcagtagacagaaataa

SEQ ID NO: 30 - protein, 409 aa, Drosophila melanogaster
MLRRAQCLLRLHGNGGHSLVSRFRNYATDEGNPKQNPNPNPRAQKPGTKNLPALRNPFAAAQDRTKNSYLTMVEIFQERDVHRRNHVEFIYAALKNMADFGVERDLEVYKALINVMPKGKFIPTNMFQAEFMHYPKQQQCIIDLLEQMEDCGVMPDHEMEAMLLNVFGRQGHPLRKYWRMMYWMPKFKNLSPWPLPDPVPDDTLEMAKLALERMCTVDLRSKITVFETSELKDAIDDTWIVSGMSPEQEKLLREHSRQKALYIEGPFHIWLRNRRINYFTLRADADSEFLSELDERQLDEDDVSHIEVPFFGRAPPRRHNQLGKLRSVHQQDDGTIMAICATGTSTKDSLLSWIRLLEANGNPSIGEVPVLFRFTSEVPAKAEEIEGGASVPATSDNSSQDEHISSRQK